(19) United States
(12) Patent Application Publication
(10) Pub. No.: US 2011/0190201 A1
(43) Pub. Date: Aug. 4, 2011

(54) Method, device, and kit for maintaining physiological levels of steroid hormone in a subject

(75) Inventors: Roderick A. Hyde, Redmond, WA (US); Muriel Y. Ishikawa, Livermore, CA (US); Dennis J. Rivet, Portsmouth, VA (US); Elizabeth A. Sweeney, Seattle, WA (US); Lowell L. Wood, JR., Bellevue, WA (US); Victoria Y.H. Wood, Livermore, CA (US)

(73) Assignee: Searete LLC, Bellevue, WA (US)

(21) Appl. No.: 13/065,046

(22) Filed: Mar. 10, 2011

Related U.S. Application Data
(62) Division of application No. 12/288,910, filed on Oct. 23, 2008, now abandoned.
Application No. 12/288,910 is a continuation-in-part of application No. 12/220,704, filed on Jul. 24, 2008.
Application No. 12/220,704 is a continuation-in-part of application No. 12/220,707, filed on Jul. 24, 2008.

(51) Int. Cl. A61K 38/22; A61P 3/00; A61P 3/10; A61K 31/565; A61K 31/568

(52) U.S. Cl. 514/6.9; 514/9.7; 514/182; 514/178

(57) ABSTRACT

A method, device, or kit is provided which maintain a substantially physiological cyclic pre-menopausal level of one or more steroid hormones in a mammalian subject in need thereof. The method, device, or kit includes providing to the subject at least one treatment regimen including replacement therapy for the one or more steroid hormones or metabolites or modulators thereof, wherein the at least one treatment regimen is determined based on pre-menopausal cyclic steroid hormone levels of the subject and on current cyclic steroid hormone levels of the subject, wherein the at least one treatment regimen is configured to maintain the subject's one or more steroid hormones or metabolites or modulators thereof at substantially physiological cyclic pre-menopausal levels. The method, device, or kit can be used for the treatment of a disease or condition in the mammalian subject.

### RELATED APPLICATIONS

For purposes of the USPTO extra-statutory requirements, the present application constitutes a continuation-in-part of U.S. patent application Ser. No. To be Assigned, entitled METHOD, DEVICE, AND KIT FOR MAINTAINING PHYSIOLOGICAL LEVELS OF STEROID HORMONE IN A SUBJECT, naming Roderick A. Hyde, Muriel Y. Ishikawa, Dennis J. Rivet, Elizabeth Sweeney, Lowell L. Wood, Jr. and Victoria Y. H. Wood as inventors, filed 24 Jul. 2008, which is currently co-pending, or is an application of which a currently co-pending application is entitled to the benefit of the filing date.

For purposes of the USPTO extra-statutory requirements, the present application constitutes a continuation-in-part of U.S. patent application Ser. No. To be Assigned, entitled SYSTEM AND DEVICE FOR MAINTAINING PHYSIOLOGICAL LEVELS OF STEROID HORMONE IN A SUBJECT, naming Roderick A. Hyde, Muriel Y. Ishikawa, Dennis J. Rivet, Elizabeth Sweeney, Lowell L. Wood, Jr. and Victoria Y. H. Wood as inventors, filed 24 Jul. 2008, which is currently co-pending, or is an application of which a currently co-pending application is entitled to the benefit of the filing date.

All subject matter of the Related Applications and of any and all parent, grandparent, great-grandparent, etc. applications of the Related Applications is incorporated herein by reference to the extent such subject matter is not inconsistent herewith.

### SUMMARY

The method described herein maintains a substantially physiological cyclic pre-menopausal level of one or more steroid hormones in a mammalian subject in need thereof. The method further maintains a substantially physiological cyclic level of one or more steroid hormones in a male mammalian subject in need thereof. The methods can be used for the treatment of a disease or condition in the mammalian subject, which includes, but is not limited to, neoplastic disease, neurologic disease, cardiovascular disease, or inflammatory disease.

The method described herein for maintaining a substantially physiological cyclic pre-menopausal level of one or more steroid hormones in a mammalian subject in need thereof comprises providing to the subject at least one treatment regimen including replacement therapy for the one or more steroid hormones or metabolites or modulators thereof, wherein the at least one treatment regimen is determined based on pre-menopausal cyclic steroid hormone levels of the subject and on current cyclic steroid hormone levels of the subject, wherein the at least one treatment regimen is configured to maintain the subject's one or more steroid hormones or metabolites or modulators thereof at substantially physiological cyclic pre-menopausal levels. The at least one treatment regimen includes a pharmaceutical composition including, but not limited to, one or more steroid hormones, metabolites, modulators, mimetics or analogs thereof. The method can further comprise determining the one or more steroid hormones levels in the subject during a treatment period. In a further aspect, the method includes at least one second treatment regimen including replacement therapy for one or more steroid hormones or one or more metabolites or modulators, thereof for maintaining a substantially physiological cyclic pre-menopausal level of one or more steroid hormones in the subject. The at least one treatment regimen can be determined based at least in part on a time-history of serum steroid hormone levels in the subject, or on inferred peak values or minimal values of serum steroid hormone levels in the subject, on age of the subject, or on categorization relative to profiles of patient populations. The at least one treatment regimen can be determined based on a genetic profile of the subject. The method described herein for restoring a physiological level of one or more steroid hormones in a mammalian subject comprises providing to the subject at least one treatment regimen including replacement therapy for the one or more steroid hormones or metabolites or modulators thereof, wherein the at least one treatment regimen is individualized and determined based on steroid hormone levels prior to disease diagnosis in the subject and based on current steroid hormone levels in the subject, wherein the at least one treatment regimen is configured to maintain the subject one or more steroid hormones or metabolites thereof at substantially physiological pre-disease levels.

A method described herein for maintaining a substantially physiological cyclic pre-menopausal level of one or more steroid hormones in a mammalian subject in need thereof comprises providing to the subject at least one treatment regimen including replacement therapy for the one or more steroid hormones or metabolites or modulators thereof, wherein the at least one treatment regimen is determined based on pre-menopausal cyclic steroid hormone levels of the subject and on current cyclic steroid hormone levels of the subject. The at least one treatment regimen can be configured to maintain the subject's one or more steroid hormones or metabolites or modulators thereof at substantially physiological cyclic pre-menopausal levels. The current cyclic steroid hormone levels can be based on one or more of peri-menopausal, early menopausal, late menopausal, or post-menopausal steroid hormone levels of the subject. The method can further comprise determining the one or more steroid hormones levels in the subject during a treatment period. The treatment period can include a time period preceding treatment or a time period during treatment with the at least one treatment regimen. In one aspect, determining the one or more steroid hormones levels can occur at multiple time points during the treatment period. The method described herein can further comprise providing to the subject at least one second treatment regimen adjusted to maintain the subject's one or more steroid hormones or one or more metabolites or modulators thereof at substantially physiological cyclic pre-menopausal levels. The at least one treatment regimen can be determined based at least in part on a time-history of serum steroid hormone levels in the subject, on inferred peak values or minimal values of serum steroid hormone levels in the subject, on age of the subject, or categorization relative to profiles of patient populations. The at least one treatment regimen can be determined based at least in part on Fourier analysis of the cyclic steroid hormone levels in the subject, or on harmonic analysis of the cyclic steroid hormone levels in the subject. The at least one treatment regimen can be determined based on a genetic profile of the subject. The method described herein can further comprise determining a genetic profile of the subject. The at least one treatment regimen can be determined based at least in part on scaled values of the pre-menopausal cyclic steroid hormone levels in the subject. The at least one treatment regimen can be determined based at least in part on a scaled value approximately equal to one. The at least one treatment regimen can be determined based at least in part on a scaled value dependent on age of the subject. In one aspect, the subject is peri-menopausal, early menopausal, late menopausal, or post menopausal.

The method described herein can further comprise determining the subject's one or more steroid hormones levels when the subject is premenopausal and has no underlying condition affecting production of steroid hormones. The at least one treatment regimen can be determined based at least in part on a time-history of serum steroid hormone levels in the subject, on inferred peak values or minimal values of serum steroid hormone levels in the subject, on age of the subject, or categorization relative to profiles of patient populations. The at least one treatment regimen can be determined based on a genetic profile of the subject. The method can further comprise determining a genetic profile of the subject. The at least one treatment regimen can be determined while the subject is not pre-menopausal. The at least one treatment regimen can be determined while the subject has an underlying condition affecting production of hormones. In one aspect, the subject has undergone oopherectomy, and the at least one treatment regimen is determined based on the subject's pre-surgical cyclic steroid hormone levels. The subject can be female. The treatment regimen can be provided orally, transdermally or subdermally. The hormones or their metabolically related compounds include, but are not limited to, sterols, androgens, progestogens, estrogens, follicle-stimulating hormone, luteinizing hormone, inhibin B, anti-Mullerian hormone thyroid-related hormones, or analogs or derivatives thereof. In one aspect, the androgen is testosterone. In a further aspect, the estrogen is an estradiol or estrone. The at least one treatment regimen can be determined based at least in part on one or more ratios of levels of the steroid hormones or metabolically related compounds in the subject, and the treatment regimen restores the one or more ratios to physiological cyclic pre-menopausal levels. The one or more ratios can include a ratio of different estradiols, an estradiol to an estrone, or a testosterone to an estrogen. The one or more ratios can include a ratio of the one or more steroid hormones or metabolically related compounds at different time intervals of the cycle in the subject. The method described herein can further comprise determining one or more ratios of levels of the steroid hormones or metabolically related compounds in the subject. The at least one treatment regimen can be determined based on the subject's pre-menopausal cyclic levels of one or more steroid hormone receptors and on the subject's current levels of the one or more steroid hormone receptors. The one or more hormone receptors include, but are not limited to, an estrogen receptor. The at least one treatment regimen can be based on diagnostic data. The diagnostic data can be based on serum levels of at least one of inflammation-related compounds, lipid-related compounds, or disease-related markers. The at least one inflammation-related compound includes, but is not limited to, C-reactive protein. The at least one lipid-related compound includes, but is not limited to, a high density lipoprotein or a low density lipoprotein. The at least one disease-related marker includes, but is not limited to, a cancer marker, a neurological disease marker, a metabolic disease marker, or a cardiovascular disease marker. The at least one treatment regimen can be determined using an algorithm designed to determine a dosage of the one or more steroid hormones or metabolites, modulators, or analogs thereof in the at least one treatment regimen. The subject's pre-menopausal cyclic steroid hormone levels or one or more of the subject's peri-menopausal, early menopausal, late menopausal, or post-menopausal steroid hormone levels can be determined for a period of time from at lease approximately 7 days to approximately 168 days.

A method described herein for maintaining a substantially physiological cyclic pre-menopausal level of one or more steroid hormones in a mammalian subject in need thereof comprise determining cyclic pre-menopausal levels of the one or more steroid hormone in the subject, and providing to the subject at least one treatment regimen including replacement therapy for the one or more steroid hormones or metabolites or modulators thereof, wherein the at least one treatment regimen is determined based on the subject's pre-menopausal cyclic steroid hormone levels and on current cyclic steroid hormone levels of the subject. The at least one treatment regimen can be configured to maintain the subject's one or more steroid hormones or metabolites or modulators thereof at substantially physiological cyclic levels. The current cyclic steroid hormone levels can be based on one or more of peri-menopausal, early menopausal, late menopausal, or post-menopausal steroid hormone levels of the subject. The method can further comprise determining at least one of peri-menopausal, early menopausal, late menopausal, or post-menopausal cyclic levels of the one or more steroid hormone in the subject. The at least one treatment regimen can be determined based on a previous time-history of serum steroid hormone levels in the subject, on inferred peak values or minimal values of serum steroid hormone levels in the subject, on age of the subject, or on categorization relative to profiles of patient populations. The at least one treatment regimen can be determined based on a genetic profile of the subject.

A method described herein for treating dementia in a mammalian subject comprises providing to the subject at least one treatment regimen including replacement therapy for the one or more steroid hormones or metabolites or modulators thereof, wherein the at least one treatment regimen is determined based on the subject's pre-menopausal cyclic steroid hormone levels and on current cyclic steroid hormone levels in the subject. The at least one treatment regimen can be configured to maintain the subject's one or more steroid hormones or metabolites or modulators thereof at substantially physiological cyclic levels in an amount effective to reduce or eliminate dementia in the subject. The current cyclic steroid hormone levels, can be based on one or more of peri-menopausal, early menopausal, late menopausal, or post-menopausal steroid hormone levels of the subject. The dementia includes, but is not limited to, Alzheimer's disease.

A method described herein for treating cardiovascular disease in a mammalian subject comprises providing to the subject at least one treatment regimen including replacement therapy for the one or more steroid hormones or metabolites or modulators thereof, wherein the at least one treatment regimen is determined based on the subject's pre-menopausal cyclic steroid hormone levels and on current cyclic steroid hormone levels. The at least one treatment regimen can be configured to maintain the subject's one or more steroid hormones or metabolites or modulators thereof at substantially physiological cyclic levels in an amount effective to reduce or eliminate cardiovascular disease in the subject. The current cyclic steroid hormone levels are based on one or more of peri-menopausal, early menopausal, late menopausal, or post-menopausal steroid hormone levels of the subject. The cardiovascular disease includes, but is not limited to, at least one of heart disease, bleeding, inflammation, atherosclerosis, acute myocardial infarction, angina pectoris, myocardial ischemia, reperfusion injury, venous thrombosis, coronary insufficiency, coronary valve disease, coronary valve stenosis, coronary heart disease, atherothrombotic stroke, or intermittent claudication.

A method described herein for treating cancer in a mammalian subject comprises providing to the subject at least one treatment regimen including replacement therapy for the one or more steroid hormones or metabolites or modulators thereof, wherein the at least one treatment regimen is determined based on the subject's pre-menopausal cyclic steroid hormone levels and on current cyclic steroid hormone levels. The at least one treatment regimen can be configured to maintain the subject's one or more steroid hormones or metabolites or modulators thereof at substantially physiological cyclic levels in an amount effective to provide at least one of a complete response, a partial response or a stable disease state in the subject. The current cyclic steroid hormone levels can be based on one or more of peri-menopausal, early menopausal, late menopausal, or post-menopausal steroid hormone levels of the subject. The cancer includes, but is not limited to, at least one of ovarian cancer, uterine cancer, uterine sarcoma, endometrial carcinoma, colorectal cancer, or lung cancer.

A method described herein for treating a metabolic disease in a mammalian subject comprises providing to the subject at least one treatment regimen including replacement therapy for the one or more steroid hormones or metabolites or modulators thereof, wherein the at least one treatment regimen is determined based on the subject's pre-menopausal cyclic steroid hormone levels and on current cyclic steroid hormone levels. The at least one treatment regimen can be configured to maintain the subject's one of more steroid hormones or metabolites or modulators thereof at substantially physiological cyclic levels in an amount effective to reduce or eliminate metabolic disease in the subject. The current cyclic steroid hormone levels can be based on one or more of peri-menopausal, early menopausal, late menopausal; or post-menopausal steroid hormone levels of the subject. The metabolic disease includes, but is not limited to, at least one of diabetes, metabolic syndrome, or thyroid disease.

A method described herein for restoring a physiological level of one or more steroid hormones in a mammalian subject can comprise providing to the subject at least one treatment regimen including replacement therapy for the one or more steroid hormones or metabolites or modulators thereof, wherein the at least one treatment regimen is determined based on the subject's pre-menopausal cyclic steroid hormone levels and on current cyclic steroid hormone levels in the subject.

The at least one treatment regimen can be configured to maintain the subject's one or more steroid hormones or metabolites or modulators thereof at substantially physiological cyclic pre-menopausal levels. The current cyclic steroid hormone levels can be based on one or more of peri-menopausal, early menopausal, late menopausal, or post-menopausal steroid hormone levels of the subject. The method can further comprise determining the one or more steroid hormones levels in the subject during a treatment period. The treatment period can include a time period preceding treatment or a time period during treatment with the at least one treatment regimen. In one aspect, determining the one or more steroid hormones levels can occur at multiple time points during the treatment period. The method described herein can further comprise providing to the subject at least one second treatment regimen adjusted to maintain the subject's one or more steroid hormones or one or more metabolites or modulators thereof at substantially physiological cyclic pre-menopausal levels. The at least one treatment regimen can be determined based at least in part on a time-history of serum steroid hormone levels in the subject, on inferred peak values or minimal values of serum steroid hormone levels in the subject, on age of the subject, or categorization relative to profiles of patient populations. The at least one treatment regimen can be determined based at least in part on Fourier analysis of the cyclic steroid hormone levels in the subject, or on harmonic analysis of the cyclic steroid hormone levels in the subject. The at least one treatment regimen can be determined based on a genetic profile of the subject. The method described herein can further comprise determining a genetic profile of the subject. The at least one treatment regimen can be determined based at least in part on scaled values of the pre-menopausal cyclic steroid hormone levels in the subject. The at least one treatment regimen can be determined based at least in part on a scaled value approximately equal to one. The at least one treatment regimen can be determined based at least in part on a scaled value dependent on age of the subject. In one aspect, the subject is peri-menopausal, early menopausal, late menopausal, or post menopausal.

A kit described herein comprises at least one treatment regimen including one or more steroid hormones or metabolites, modulators, or analogs thereof providing varying dosages of the one or more steroid hormones or metabolites, modulators, or analogs thereof, the at least one treatment regimen based on the subject's pre-menopausal cyclic steroid hormone levels and on current cyclic steroid hormone levels, and instructions for administering the at least one treatment regimen and for monitoring the effectiveness of hormone replacement therapy in the menopausal subject.

The at least one treatment regimen can be configured to maintain a substantially physiological cyclic pre-menopausal level of one or more steroid hormones in a mammalian subject in need thereof. The current cyclic steroid hormone levels can be based on one or more of peri-menopausal, early menopausal, late menopausal, or post-menopausal steroid hormone levels of the subject. The varying dosages can be based on the subject's current cyclic steroid hormone levels and the substantially physiological cyclic pre-menopausal steroid hormone levels. The varying dosages can be based on a time-history of serum steroid hormone levels in the subject, on inferred peak values or minimal values of serum steroid hormone levels in the subject, on age of the subject, or on categorization relative to profiles of patient populations. The varying dosages can be based on a genetic profile of the subject. The at least one treatment regimen can provide varying dosages on a periodic basis to maintain the subject's substantially physiological cyclic steroid hormone levels. The periodic basis can be daily, weekly, or every 28 days.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

FIGS. 1A and 1B depict a diagrammatic view of one aspect of an exemplary embodiment of a method for maintaining a substantially physiological cyclic pre-menopausal level of one or more steroid hormones in a mammalian subject in need thereof.

FIG. 2 depicts a logic flowchart of a method.

FIG. 3 depicts a logic flowchart of a method.

FIG. 4 depicts a logic flowchart of a method.

FIG. 5 depicts a logic flowchart of a method.

FIG. 6 depicts a logic flowchart of a method.

FIG. 7 depicts some aspects of a system that may serve as an illustrative environment for subject matter technologies.

FIG. 8 depicts some aspects of a system that may serve as an illustrative environment for subject matter technologies.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

The present application uses formal outline headings for clarity of presentation. However, it is to be understood that the outline headings are for presentation purposes, and that different types of subject matter may be discussed throughout the application (e.g., method(s) may be described under composition heading(s) and/or kit headings; and/or descriptions of single topics may span two or more topic headings). Hence, the use of the formal outline headings is not intended to be in any way limiting.

The method described herein maintains a substantially physiological cyclic pre-menopausal level of one or more steroid hormones in a mammalian subject in need thereof. The method further maintains a substantially physiological cyclic level of one or more steroid hormones in a male mammalian subject in need thereof. The methods can be used for the treatment of a disease or condition in the mammalian subject, which includes, but is not limited to, neoplastic disease, neurologic disease, metabolic disease, cardiovascular disease, or inflammatory disease.

Hormone replacement or supplemental therapy has been used for some time to relieve symptoms of menopause or to provide protection from disorders such as osteoporosis. However, early and more recent studies have offered evidence that treatment with exogenous hormones carries risks, and limits have been suggested for treatments, including those on dosages and formulations. While incorporating these limitations, current therapies are still designed based on population data, with discussions on the need for individualized treatment regimens limited to health status and disease state without regard for individual medical history data on hormonal levels (for example, see Notelovitz, General Medicine, 8: 84, 2006. The Biologic and Pharmacologic Principles for Age-Adjusted Long-term Estrogen Therapy). Such proposals still rely on population-based “normal” ranges for hormone levels. In fact, levels of steroid hormones can differ greatly among individuals, and can be greatly affected by multiple factors including race, environment, and genotypes (for examples see Ellison et al., Lancet 342: 433-434, 1993; Pinheiro et al., Cancer Epidemiology Biomarkers & Prevention 14: 2147-2153, 2005; Nùñez-de la Mora et al., PLoS Med 4(5): e167 2007; Jasienka, et al., Cancer Epidemiology, Biomarkers and Prevention 15: 2131-2135, 2006; Small, et al., Human Reproduction 20(8): 2162-2167, 2005; and Sharp et al., Am J Epidemiol 160: 729-740, 2004; which are incorporated herein by reference). Thus, regimens designed using population-based levels in many cases may be inappropriate for a patient, providing too much, too little, or the wrong type(s) of steroid hormones, potentially resulting in ineffectual or even harmful outcomes. In addition, much attention is now focusing on hormone treatment in younger women, such as women transitioning into and through natural menopause or women with a loss of ovarian function due to surgery, exposure, or disease. Studies in humans and animals provide evidence that pre-menopausal exposure and/or higher lifetime exposure to hormones confers protection against neurological disease (see, e.g., McLay, et al., J. Neuropsychiatry Lin. Neurosci. 15:161-167, 2003; Suzuki et al. PNAS USA, 104: 6013-6018, 2007; Ryan, et al. Int. Psychogeriatr. 20:47-56, 2008; and Morrison, et al., J. Neurosci. 26:10332-10348, 2006; which are incorporated herein by reference) and cardiovascular disease (van der Schouw et al., Lancet 16:714-8 1996). Current therapy and clinical trials (e.g., The Kronos Early Estrogen Prevention Study (KEEPS)) are now focusing on treating women transitioning into menopause and early menopause with estrogens, alone or in combination, administered orally or transdermally (see, e.g., Clarkson, Menopause 14: 373-84, 2007; Harman, et al., Climacteric 8(1):3-12, 2005; Qiao, et al. in Gender Medicine. 5 Suppl. A, S46-S64, 2008, which are incorporated herein by reference).

The methods described herein maintain a substantially physiological cyclic level of one or more steroid hormones in a mammalian subject in need thereof which includes an individualized treatment regimen for the subject. The individualized treatment regimen includes replacement therapy for one or more steroid hormones, or metabolites or modulators thereof. The at least one treatment regimen includes a pharmaceutical composition of one or more steroid hormones, or metabolites, modulators, mimetics or analogs thereof. The treatment regimen can be based upon information derived from pre-menopausal hormone levels or pre-disease hormone levels in the subject. In this context, a physiological level of a hormone includes the level of hormone measured at a given time. A physiological premenopausal level can be a level of the hormone as measured at a point in time during premenopause in a female subject. A physiological pre-disease level can be a level of the hormone as measured at a point in time prior to occurrence of disease or prior to surgery to treat a disease in a female or male subject. A current physiological level can be the level of the hormone as measured just prior to determining a treatment regimen. The physiological levels of the one or more hormones of the female subject may be provided by collected measurements or provided as part of the subject's medical history, and the physiological premenopausal levels may include cyclic and/or temporal, e.g., age-related or weight-related, variations. A treatment regimen can be determined based on the physiological premenstrual levels and the current physiological levels. The determined treatment regimen may, for example, include maintaining physiological pre-menopausal hormone levels throughout perimenopause, menopause and/or postmenopause by administration of one or more exogenous hormones, metabolites, modulators, or related compounds or analogs thereof, and may include continual, cyclical, or time-dependent administration.

Determining a physiological level of a hormone may be based upon recurrent measurements of pre-menopausal or pre-disease hormone levels in the subject which may be used to provide at least one treatment regimen to the subject including replacement therapy for the one or more steroid hormones, or metabolites or modulators thereof. The physiological pre-menopausal hormone levels or physiological pre-disease hormone levels in the subject can be obtained from past medical history, e.g., information from a past medical history provided by the subject, or present medical evaluation, e.g., information from current measurements by assay for pre-menopausal hormone levels or pre-disease hormone levels, or a combination thereof.

Prior to determining a treatment regimen, additional information regarding the physiological status of the subject may be gathered and assessed. For example, information on the subject's own history or his or her family's history of diseases, including genetic information, may be collected. The medical evaluation can include a genetic profile of the subject regarding genes, genetic mutations, or genetic polymorphisms that may indicate risk factors that affect disease and/or are related to steroid hormone levels, hormone receptors, modulators (e.g., agonists or antagonists, of steroid hormones or steroid hormone receptors), enzymes involved in steroidogenesis, metabolites, or analogs thereof, or factors causing genetic disease or a genetic predisposition to disease in the subject. Examples of enzymes involved in steroidogenesis include, but are not limited to, biosynthetic enzymes for hormones or hormone receptor synthesis, e.g., CYP 17. Medical evaluation regarding genetic profiling or genetic testing can be provided as a current determination of genetic risk factors, or as part of the subject's medical history. Genetic profiling or genetic testing can be used to design a treatment regimen and thus determine an optimal level individualized for the subject of the one or more steroid hormones, steroid hormone receptors, metabolites, or modulators or analogs thereof, obtained during a pre-menopausal or pre-disease period from the subject. A physician may use the genetic profiling or genetic testing information to determine a genetic basis for needed treatment to maintain a substantially physiological cyclic level of one or more steroid hormones in a mammalian subject in need thereof.

Prior to determining a treatment regimen, additional information regarding diseases and possible therapeutic treatment contained in population databases may be gathered and assessed. The medical evaluation can include information in a population database on disease risks, available drugs and formulations, and documented population responses to drugs and formulations.

A system is described which comprises at least one computer program included on computer-readable medium for use with at least one computer system and wherein the computer program includes a plurality of instructions including one or more instructions for measuring pre-menopausal cyclic steroid hormone levels or measuring steroid hormone levels prior to disease diagnosis in a mammalian subject, one or more instructions for measuring current cyclic steroid hormone levels in the mammalian subject, and one or more instructions for determining at least one treatment regimen including replacement therapy for one or more steroid hormones, or metabolites or modulators thereof, based on the steroid hormone levels prior to onset of menopause or prior to disease diagnosis in the subject and based on the current steroid hormone levels in the mammalian subject. A device is described which incorporates the system programmed to maintain physiological cyclic levels of one or more steroid hormones or metabolites thereof in a mammalian subject in need thereof.

The methods described herein maintain a substantially physiological cyclic level of one or more steroid hormones in a mammalian subject in need thereof (e.g., pre-menopausal level in a female subject) based upon transitional changes in the levels of the one or more steroid hormones in the subject. Such changes indicate a need for a treatment regimen including replacement therapy for the one or more steroid hormones, or metabolites or modulators thereof, to offset a decrease in the level of the one or more steroid hormones in the mammalian-subject. The change in levels of the one or more steroid hormones can occur as a result of perimenopause, menopause, or postmenopause resulting in decreased levels of steroid hormones in a female subject. The changes in levels of the one or more steroid hormones can occur as a result of surgery (e.g., oophorectomy, ovariectomy, or orchiectomy), damage (e.g., loss of function due to radiation or chemical exposure), or disease, (e.g., cancer, inflammatory disease, or cardiovascular disease) in a female subject or a male subject. In a further aspect, the method can restore a balance of the one or more steroid hormones in a subject in need thereof. The method includes, but is not limited to, providing to a subject at least one treatment regimen to maintain a substantially physiological cyclic pre-menopausal level of one or more steroid hormones (e.g., a pre-menopausal level in a female subject) that closely mimics naturally cyclical dosage in female or male subjects to treat or prevent diseases associated with reduced levels of steroid hormones in the subject.

The method described herein for maintaining a substantially physiological cyclic pre-menopausal level of one or more steroid hormones in a mammalian subject in need thereof comprises providing to the subject at least one treatment regimen including replacement therapy for the one or more steroid hormones, or metabolites or modulators thereof, wherein the at least one treatment regimen is determined based on pre-menopausal cyclic steroid hormone levels of the subject and on current cyclic steroid hormone levels of the subject, wherein the at least one treatment regimen is configured to maintain the subject's one or more steroid hormones or metabolites thereof at substantially physiological cyclic pre-menopausal levels. The method can further comprise determining the one or more steroid hormones levels in the subject during a treatment period. In a further aspect, the method includes at least one second treatment regimen to the subject adjusted to maintain the subject's one or more steroid hormones or one or more metabolites or modulators thereof at substantially physiological cyclic pre-menopausal levels. The at least one treatment regimen can be determined based at least in part on a time-history of serum steroid hormone levels in the subject, on inferred peak values or minimal values of serum steroid hormone levels in the subject, on age of the subject, or on categorization relative to profiles of patient populations. The at least one treatment regimen can be determined based on a genetic profile of the subject.

The method described herein for restoring a physiological level of one or more steroid hormones in a mammalian subject comprises providing to the subject at least one treatment regimen including replacement therapy for the one or more steroid hormones or metabolites or modulators thereof, wherein the at least one treatment regimen is determined based on steroid hormone levels prior to disease diagnosis in the subject and on current steroid hormone levels in the subject, wherein the at least one treatment regimen is configured to maintain the subject's one or more steroid hormones or metabolites or modulators thereof at substantially physiological pre-disease levels.

Cyclical serum levels of steroid hormones include the serum levels over a period of time such as a menstrual cycle or a 28-day cycle, including the changes in levels during that time. Optimum cyclical serum levels includes the optimum changes in the levels during a period of time such as a menstrual cycle or a 28-day cycle.

Replacement therapy includes a treatment for a disease or condition in a mammalian subject in need thereof which includes a pharmaceutical composition of one or more steroid hormones or metabolites, modulators, mimetics, or analogs thereof. The treatment aims to maintain a substantially physiological level of one or more steroid hormones or metabolites or modulators thereof in a male subject. The treatment further aims to maintain a substantially physiological cyclic pre-menopausal level of one or more steroid hormones or metabolites or modulators thereof in a female subject. At least one treatment regimen including replacement therapy for the one or more steroid hormones or metabolites or modulators thereof includes an individualized treatment for a disease or condition and maintains a substantially physiological cyclic pre-menopausal level of the one or more steroid hormones in a mammalian subject in need thereof. The at least one treatment regimen including replacement therapy includes a pharmaceutical composition of one or more of the compounds or compositions as described herein, including but not limited to, natural or synthetic compounds with estrogenic activity; synthetic steroidal compounds having estrogenic activity; synthetic non-steroidal compounds having estrogenic activity; plant-derived phytoestrogens having estrogenic activity; esters, conjugates or prodrugs of suitable estrogens; androgens; modulators, including but not limited to selective estrogen receptor modulators (SERMs) and modulators of metabolic and/or synthetic pathways such as'enzyme regulators; and modulators of signaling pathways, progesterones; natural or synthetic compounds having progestational activity; gonadotropin hormones; or analogs, metabolites, hormone precursors, metabolite precursors, biosynthetic enzymes, DNA encoding biosynthetic enzymes, or derivatives thereof. The compound or composition further includes analogs, peptide mimetics, DNA encoding polypeptides of interest, or small chemical molecular mimetics of the one or more steroid hormones, or metabolites or modulators. The treatment aims to maintain a substantially physiological cyclic pre-menopausal level of one or more steroid hormones or metabolites thereof in a female subject. The treatment further aims to maintain a substantially physiological level of one or more steroid hormones or metabolites thereof in a male subject

Modulators include activators and inhibitors. Modulators can increase or decrease hormones or other intermediates or receptors in a manner that regulates or increase steroid hormone levels. The modulator can be a physiologic modulator or a synthetic modulator. Activators are agents that, e.g., bind to, stimulate, increase, open, activate, facilitate, enhance activation, sensitize or up regulate the activity of steroid hormones or steroid hormone receptors, e.g., agonists. Inhibitors are agents that, e.g., bind to, partially or totally block stimulation, decrease, prevent, delay activation, inactivate, desensitize, or down regulate the activity of a steroid hormone intermediate, a receptor, or a steroid hormone receptor, e.g., antagonists. Modulators include agents that, e.g., alter the interaction of the steroid hormone or steroid hormone receptor with: proteins that bind activators or inhibitors, receptors, including proteins, peptides, lipids, carbohydrates, polysaccharides, or combinations of the above, e.g., lipoproteins, glycoproteins, and the like. Modulators include genetically modified versions of naturally-occurring steroid hormones or other steroid hormone receptor ligands, e.g., with altered activity, as well as naturally occurring and synthetic ligands, antagonists, agonists, small chemical molecules and the like.

A treatment regimen includes a therapeutic amount of one or more steroid hormones, or metabolites, modulators, or analogs thereof in a pharmaceutical composition. The treatment regimen further includes the schedule of changes in the dosage of the pharmaceutical composition to maintain a substantially physiological cyclical serum level individualized for the subject. Treating or treatment includes the administration of the one or more steroid hormones, or metabolites, modulators, or analogs thereof, to prevent or delay the onset of the symptoms, complications, or biochemical indicia of a disease, alleviating the symptoms or arresting or inhibiting further development of the disease, condition, or disorder, e.g., neoplastic disease, neurologic disease, cardiovascular disease, metabolic disease, or inflammatory disease. Treatment can be prophylactic to prevent or delay the onset of the disease, or ° to prevent the manifestation of clinical or subclinical symptoms thereof, or therapeutic suppression or alleviation of symptoms after the manifestation of the disease.

A mammalian subject includes, for example, a human, a non-human primate, as well as experimental animals such as rabbits, rats, mice, sheep, dogs, cats, cows, and other animals. A mammalian subject further includes, for example, a pet, experimental animals, livestock, zoo animals, or animals in the wild. A treatment regimen including one or more steroid hormones, or metabolites, modulators, or analogs thereof, may be continuous and uninterrupted which indicates that there is no break in the treatment regimen, during the treatment period. Thus, continuous, uninterrupted administration of a combination, indicates that the combination may be administered during the entire treatment period, e.g., at least once daily or on a continuous and uninterrupted basis. The treatment regimen may be given to maintain a therapeutic level or a determined cyclic level of the one or more steroid hormones, or metabolites, modulators, or analogs thereof. The treatment regimen may be provided to the subject by transdermal, subcutaneous, parenteral or oral administration. It is expected that the treatment period for the treatment regimen of one or more steroid hormones, or metabolites, modulators, or analogs thereof will be for at least 30 days, preferably 120 days, and most preferably as long term treatment, and possibly indefinite, as one of the primary reasons for administering one or more steroid hormones or metabolites thereof is to treat a disease associated with a decrease or absence of the one or more steroid hormones in the subject. Treatment periods also may vary depending on the symptoms to be treated. Physician evaluation along with patient interaction will assist the determination of the duration of treatment. For the treatment of cancer, neurologic disease, cardiovascular disease, metabolic disease, or inflammatory disease, or reduction in symptoms thereof, it is envisioned that the treatment period could last from six months to a number of years, or indefinitely. Physician evaluation along with patient interaction will assist the determination of the duration of treatment. The administration of the treatment regimen including replacement therapy for one or more steroid hormones, or metabolites or modulators' thereof to a subject may need to be adjusted. Adjustments in the treatment regimen may depend upon the individual's medical history and fluctuations in current levels of steroid hormones in the subject. Administration of the treatment regimen may be adjusted to achieve the desired effect during a treatment period. Administration of one or more steroid hormones may be short term treatments or treatments of a finite term, that may be less than the 30 day treatment period. It is anticipated that a patient may miss, or forget to take, one or a few dosages during the course of a treatment regimen, however, such patient is still considered to be receiving continuous, uninterrupted administration.

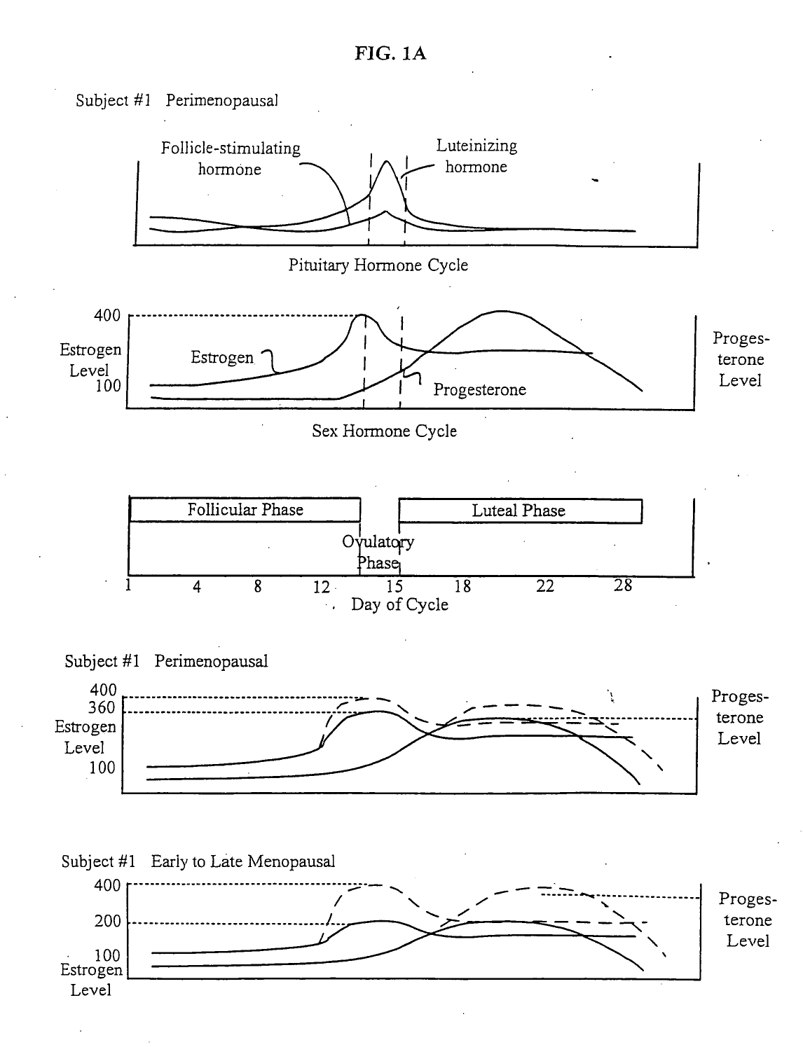

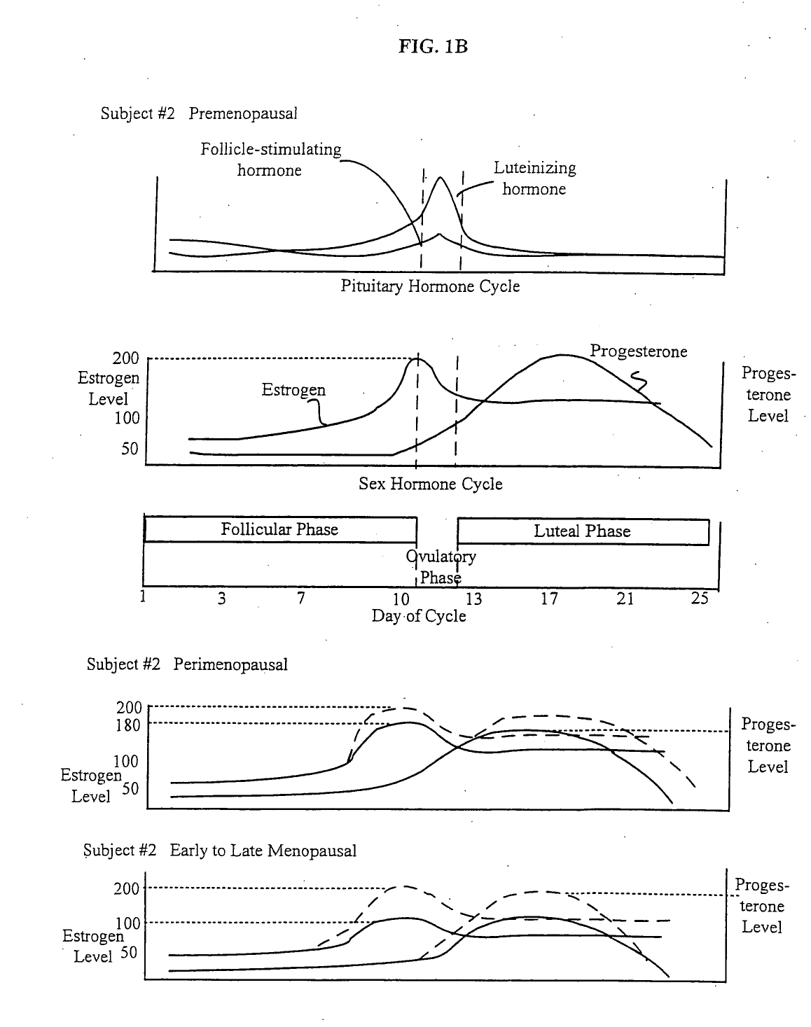

FIGS. 1A and 1B depict a diagrammatic view of an exemplary aspect of the methods and systems as described herein. The methods described herein for maintaining a substantially physiological cyclic pre-menopausal level of one or more steroid hormones in a mammalian subject in need thereof are individualized for a mammalian subject #1 (FIG. 1A) or for a mammalian subject #2 (FIG. 1B).

Female subject #1 has cyclic levels of steroid hormones, e.g., follicle stimulating hormone, luteinizing hormone, estrogen, and progesterone over a time period of 28 days. See solid lines on graph in FIG. 1A. Female subject #1 in a perimenopausal condition has current cyclic levels of estrogen and progesterone reduced. Cyclic levels of estrogen and progesterone are further reduced in subject #1′ in an early to late menopausal condition. See solid lines on graph in FIG. 1A. The method maintains a substantially physiological cyclic pre-menopausal level of one or more steroid hormones in the subject by providing to the subject at least one treatment regimen including replacement therapy for the one or more steroid hormones or metabolites or modulators thereof, wherein the at least one treatment regimen is determined based on pre-menopausal cyclic steroid hormone levels of the subject and on current cyclic steroid hormone levels of the subject. In this case, the at least one treatment regimen including replacement therapy for the one or more steroid hormones or metabolites or modulators thereof is individualized and supplements the levels of estrogen and progesterone in the subject #1 to obtain pre-menopausal hormone levels. See dashed lines on graph in FIG. 1A. Female subject #2 has cyclic levels of steroid hormones, e.g., follicle stimulating hormone, luteinizing hormone, estrogen, and progesterone over a time period of 25 days. See solid lines on graph in FIG. 1B. Female subject #2 has current cyclic levels of estrogen and progesterone reduced in a perimenopausal condition and current cyclic levels of estrogen and progesterone further reduced in an early to late menopausal condition. See solid lines on graph in FIG. 1B. The method maintains a substantially physiological cyclic pre-menopausal level of one or more steroid hormones in the subject by providing to the subject at least one treatment regimen including replacement therapy for the one or more steroid hormones or metabolites or modulators thereof, wherein the at least one treatment regimen is determined based on pre-menopausal cyclic steroid hormone levels of the subject and on current cyclic steroid hormone levels of the subject. In this case, the at least one treatment regimen including replacement therapy for the one or more steroid hormones or metabolites or modulators thereof is individualized and supplements the levels of estrogen and progesterone in the subject #2 to obtain pre-menopausal hormone levels. See dashed lines on graph in FIG. 1B.

### Operations and Processes

Following are a series of flowcharts depicting implementations of processes. The flowcharts are organized such that the initial flowcharts present implementations via an overall “big picture” or “top-level” viewpoint, and thereafter the subsequent flowcharts present alternate implementations and/or expansions of the “big picture” flowcharts as either sub-steps or additional steps building on one or more earlier-presented flowcharts. Those having ordinary skill in the art will appreciate that the style of presentation utilized herein (e.g., beginning with a presentation of a flowchart(s) presenting an overall view and thereafter providing additions to and/or further details in subsequent flowcharts) generally allows for a more rapid and reliable understanding of the various process implementations.

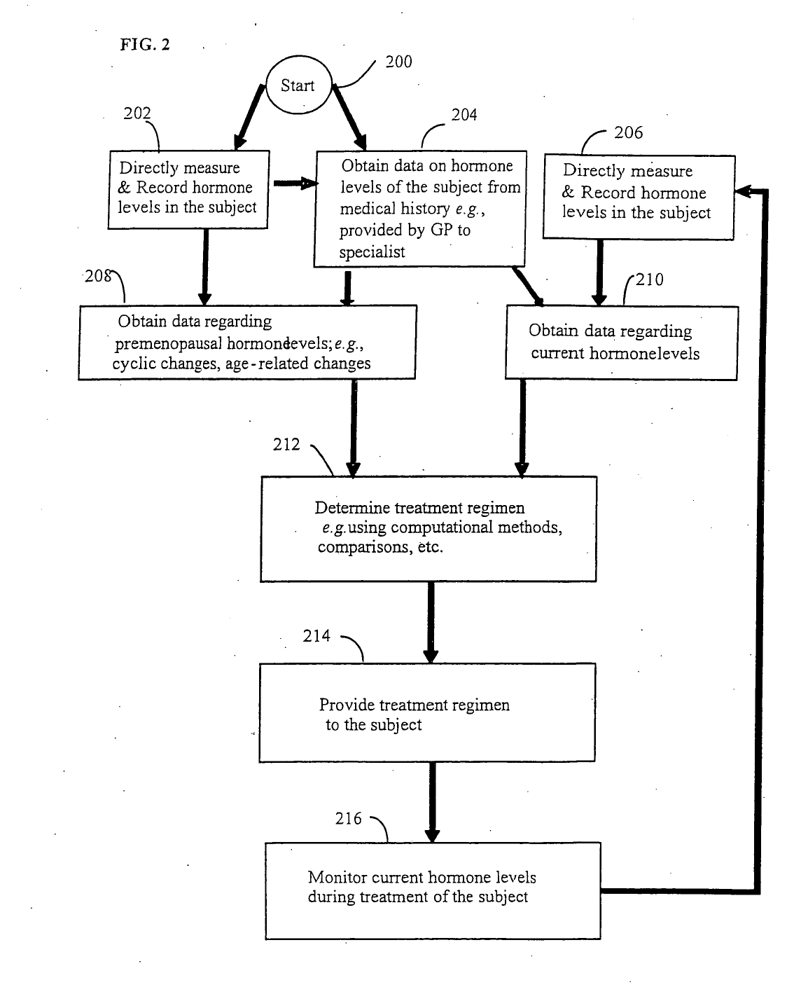

With reference to FIG. 2, depicted is a high-level logic flowchart of a process. Method step 200 shows the start of the process. Method step 202 depicts directly measuring and recording hormone levels in the subject. Method step 204 depicts obtaining data regarding hormone levels from a medical history of the subject. Method step 208 depicts obtaining data regarding premenopausal hormone levels in the subject from method steps 202 and/or 204. This data may reflect, e.g., cyclic hormonal changes or age-related hormonal changes in the subject. Method step 206 depicts directly measuring and recording hormone levels in the subject wherein the subject may be premenopausal, perimenopausal, early or late menopausal, or post menopausal. Method step 210 depicts obtaining data regarding current hormone levels from method steps 204 and/or 206. Method step 212 depicts determining a treatment regimen using methods e.g., including, but not limited to, computational methods or comparison methods. Method step 214 depicts providing at least one treatment regimen including replacement therapy for the one or more steroid hormones or metabolites or modulators thereof, to the subject. Method step 216 depicts monitoring current hormone levels during treatment of the subject. Method step 206 depicts directly measuring and recording hormone levels, e.g., during treatment of the subject. Method step 210 depicts obtaining data regarding current hormone levels. The data regarding current hormone levels is obtained from directly measuring and recording 206 current hormone levels during treatment of the subject and/or from obtaining data 204 on hormone levels from a medical history of the subject. The data is used to determine the proper treatment regimen 212 and alter or adjust the treatment regimen as needed, and providing the treatment regimen 214 to the subject. In an embodiment, method steps 202, 204, 206, 208, 210, 212, 214, and/or 216 may include accepting input related to, for example, directly measuring and recording hormone levels in the subject, obtaining data on hormone levels from medical history of the subject, determining a treatment regimen, providing a treatment regimen and monitoring current hormone levels during treatment of the subject.

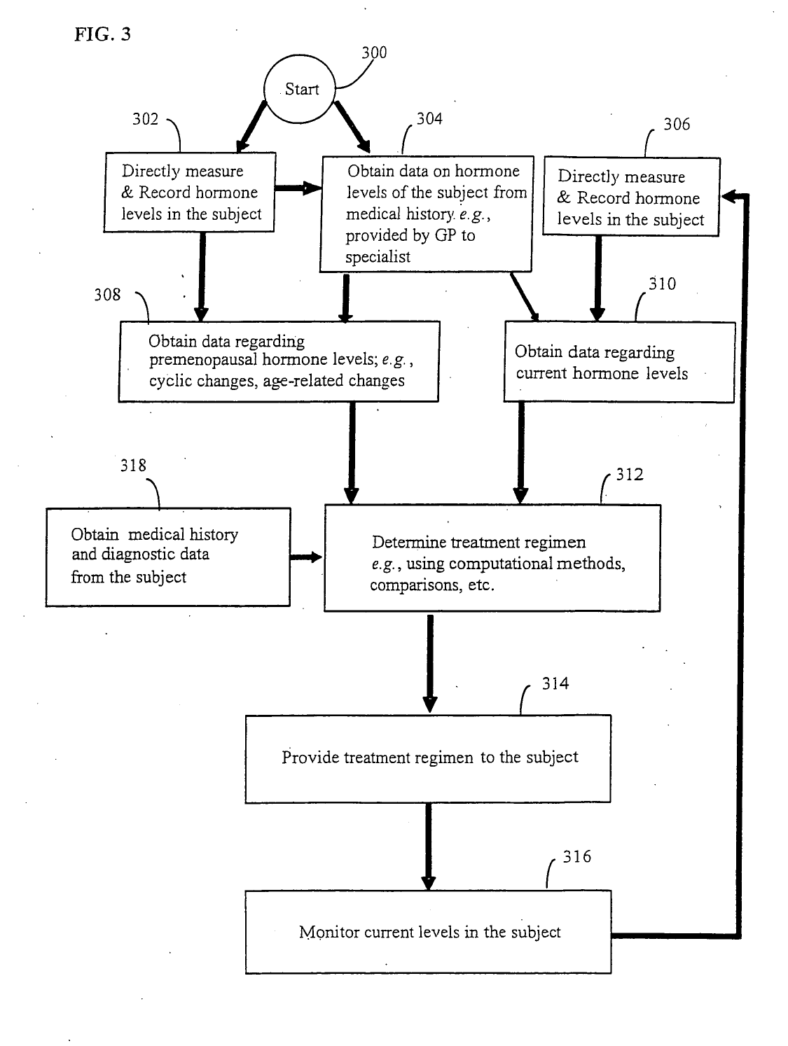

With reference to FIG. 3, depicted is a high-level logic flowchart of a process. Method step 300 shows the start of the process. Method step 302 depicts directly measuring and recording hormone levels in the subject. Method step 304 depicts obtaining data regarding hormone levels from a medical history of the subject. Method step 308 depicts obtaining data regarding premenopausal hormone levels in the subject from method steps 302 and/or 304. This data may reflect, e.g., cyclic hormonal changes or age-related hormonal changes in the subject. Method step 306 depicts directly measuring and recording hormone levels in the subject wherein the subject may be premenopausal, perimenopausal, early or late menopausal, or post menopausal. Method step 310 depicts obtaining data regarding current hormone levels from method steps 304 and/or 306. Method step 318 depicts obtaining medical history and diagnostic data from the subject. The physiological pre-menopausal hormone levels or physiological pre-disease hormone levels in the subject can be obtained from past medical history, e.g., information from a past medical history provided by the subject, or present medical evaluation, e.g., information from current measurements by assay for pre-menopausal hormone levels or pre-disease hormone levels, or a combination thereof. The medical history and diagnostic data may be used to determine a treatment regimen. Method step 312 depicts determining a treatment regimen using methods e.g., including, but not limited to, computational methods or comparison methods. Method step 314 depicts providing at least one treatment regimen including replacement therapy for the one or more steroid hormones or metabolites or modulators thereof, to the subject. Method step 316 depicts monitoring current hormone levels during treatment of the subject. Method step 306 depicts directly measuring and recording hormone levels, e.g., during treatment of the subject. Method step 310 depicts obtaining data regarding current hormone levels. The data regarding current hormone levels is obtained from directly measuring and recording 306 current hormone levels during treatment of the subject and/or from obtaining data 304 on hormone levels from a medical history of the subject. The data is used to determine the proper treatment regimen 312 and alter or adjust the treatment regimen as needed, and providing the treatment regimen 314 to the subject. In an embodiment, method steps 302, 304, 306, 308, 310, 312, 314, 316, and/or 318 may include accepting input related to, for example, directly measuring and recording hormone levels in the subject, obtaining data on hormone levels from medical history of the subject, determining a treatment regimen, providing a treatment regimen and monitoring current hormone levels during treatment of the subject.

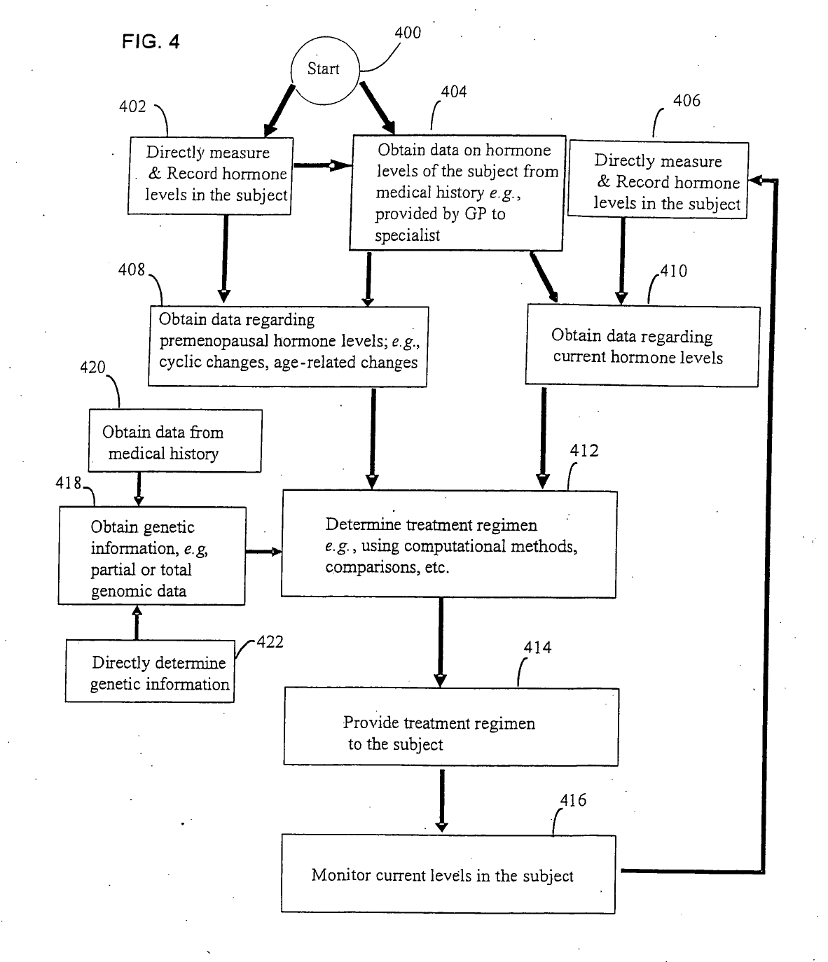

With reference to FIG. 4, depicted is a high-level logic flowchart of a process. Method step 400 shows the start of the process. Method step 402 depicts directly measuring and recording hormone levels in the subject. Method step 404 depicts obtaining data regarding hormone levels from a medical history of the subject. Method step 408 depicts obtaining data regarding premenopausal hormone levels in the subject from method steps 402 and/or 404. This data may reflect, e.g., cyclic hormonal changes or age-related hormonal changes in the subject. Method step 406 depicts directly measuring and recording hormone levels in the subject wherein the subject may be premenopausal, perimenopausal, early or late menopausal, or post menopausal. Method step 410 depicts obtaining data regarding current hormone levels from method steps 404 and/or 406. Method step 418 depicts obtaining genetic information, e.g., partial or total genomic data, from a medical history 420 of the subject or from a direct determination of genetic information 422 from the subject. The genetic information may be used to determine a treatment regimen. Method step 412 depicts determining a treatment regimen using methods e.g., including, but not limited to, computational methods or comparison methods. Method step 414 depicts providing at least one treatment regimen including replacement therapy for the one or more steroid hormones or metabolites or modulators thereof, to the subject. Method step 416 depicts monitoring current hormone levels during treatment of the subject. Method step 406 depicts directly measuring and recording hormone levels, e.g., during treatment of the subject. Method step 410 depicts obtaining data regarding current hormone levels. The data regarding current hormone levels is obtained from directly measuring and recording 406 current hormone levels during treatment of the subject and/or from obtaining data 404 on hormone levels from a medical history of the subject. The data is used to determine the proper treatment regimen 412 and alter or adjust the treatment regimen as needed, and providing the treatment regimen 414 to the subject. In an embodiment, method steps 402, 404, 406, 408, 410, 412, 414, 416, 418, 420, and/or 422 may include accepting input related to, for example, directly measuring and recording hormone levels in the subject, obtaining data on hormone levels from medical history of the subject, determining a treatment regimen, providing a treatment regimen and monitoring current hormone levels during treatment of the subject.

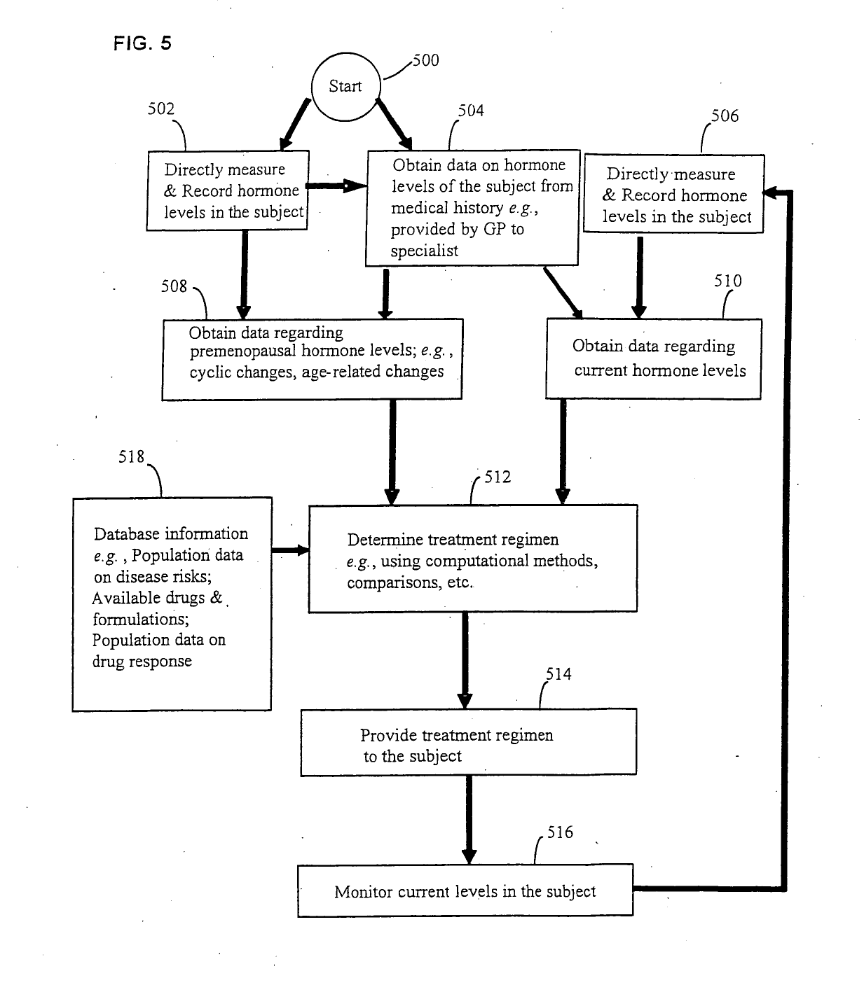

With reference to FIG. 5, depicted is a high-level logic flowchart of a process. Method step 500 shows the start of the process. Method step 502 depicts directly measuring and recording hormone levels in the subject. Method step 504 depicts obtaining data regarding hormone levels from a medical history of the subject. Method step 508 depicts obtaining data regarding premenopausal hormone levels in the subject from method steps 502 and/or 504. This data may reflect, e.g., cyclic hormonal changes or age-related hormonal changes in the subject. Method step 506 depicts directly measuring and recording hormone levels in the subject wherein the subject may be premenopausal, perimenopausal, early or late menopausal, or post menopausal. Method step 510 depicts obtaining data regarding current hormone levels from method steps 504 and/or 506. Method step 518 depicts obtaining database information, e.g., population data on disease risks, available drugs and formulations thereof, and/or population data on drug response which may be used to determine a treatment regimen. Method step 512 depicts determining a treatment regimen using methods, e.g., including, but not limited to, computational methods or comparison methods. Method step 514 depicts providing at least one treatment regimen including replacement therapy for the one or more steroid hormones or metabolites or modulators thereof, to the subject. Method step 516 depicts monitoring current hormone levels during treatment of the subject. Method step 506 depicts directly measuring and recording hormone levels, e.g., during treatment of the subject. Method step 510 depicts obtaining data regarding current hormone levels. The data regarding current hormone levels is obtained from directly measuring and recording 506 current hormone levels during treatment of the subject and/or from obtaining data 504 on hormone levels from a medical history of the subject. The data is used to determine the proper treatment regimen 512 and alter or adjust the treatment regimen as needed, and providing the treatment regimen 514 to the subject. In an embodiment, method steps 502, 504, 506, 508, 510, 512, 514, 516, and/or 518 may include accepting input related to, for example, directly measuring and recording hormone levels in the subject, obtaining data on hormone levels from medical history of the subject, determining a treatment regimen, providing a treatment regimen and monitoring current hormone levels during treatment of the subject.

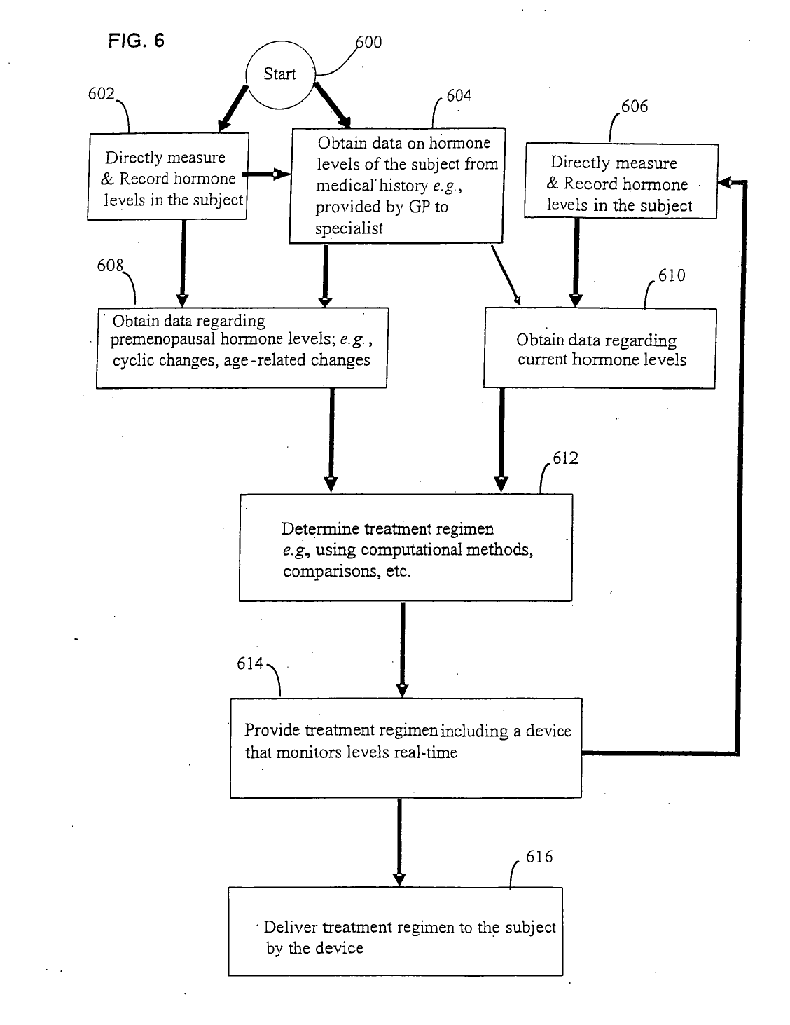

With reference to FIG. 6, depicted is a high-level logic flowchart of a process. Method step 600 shows the start of the process. Method step 602 depicts directly measuring and recording hormone levels in the subject. Method step 604 depicts obtaining data regarding hormone levels from a medical history of the subject. Method step 608 depicts obtaining data regarding premenopausal hormone levels in the subject from method steps 602 and/or 604. This data may reflect, e.g., cyclic hormonal changes or age-related hormonal changes in the subject. Method step 606 depicts directly measuring and recording hormone levels in the subject wherein the subject may be premenopausal, perimenopausal, early or late menopausal, or post menopausal. Method step 610 depicts obtaining data regarding current hormone levels from method steps 604 and/or 606. Method step 612 depicts determining a treatment regimen using methods e.g., including, but not limited to, computational methods or comparison methods. Method step 614 depicts providing at least one treatment regimen including replacement therapy for the one or more steroid hormones or metabolites or modulators thereof, to the subject, and includes a device that monitors levels real-time. Method step 616 depicts delivering the treatment regimen to the subject by the device. Method step 606 depicts directly measuring and recording hormone levels, e.g., by real time monitoring by the device 614 during treatment of the subject. Method step 610 depicts obtaining data regarding current hormone levels. The data regarding current hormone levels is obtained from directly measuring and recording 606 current hormone levels during treatment of the subject and/or from obtaining data 604 on hormone levels from a medical history of the subject. The data is used to determine the proper treatment regimen 612 and alter or adjust the treatment regimen as needed, and providing the treatment regimen 614 to the subject. In an embodiment, method steps 602, 604, 606, 608, 610, 612, 614, and/or 616 may include accepting input related to, for example, directly measuring and recording hormone levels in the subject, obtaining data on hormone levels from medical history of the subject, determining a treatment regimen, providing a treatment regimen and monitoring current hormone levels during treatment of the subject.

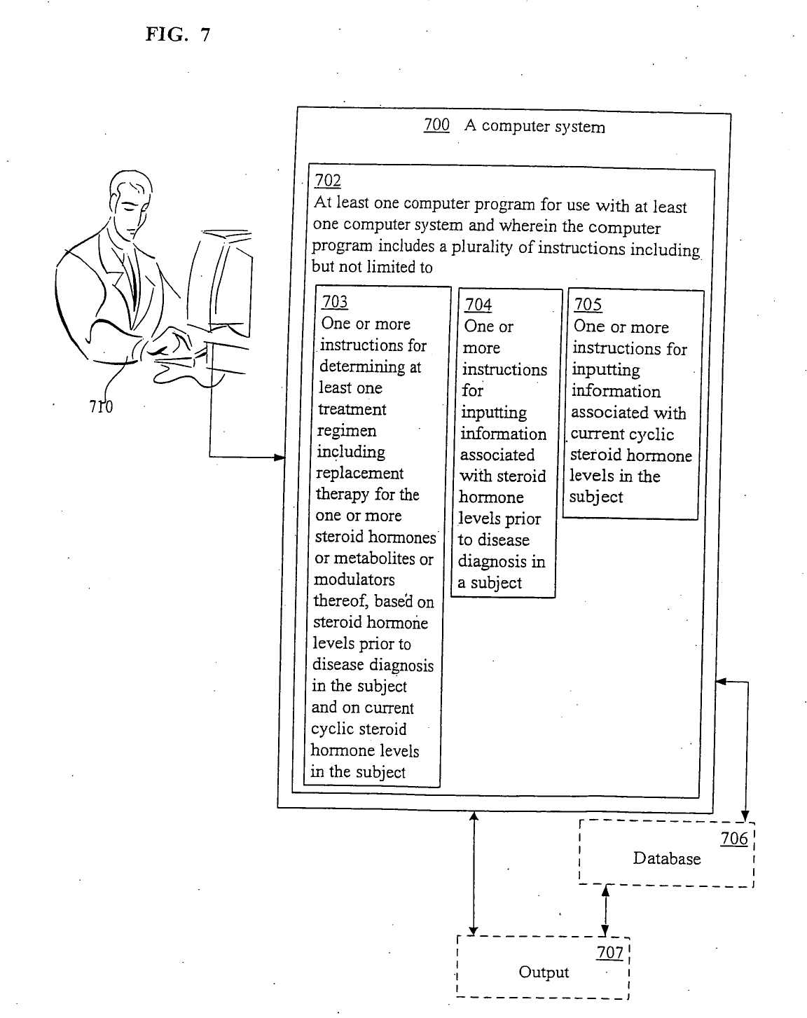

FIG. 7 depicts some aspects of a system that may serve as an illustrative environment for subject matter technologies. A system 700 may comprise at least one computer program 702 for use with at least one computer system and wherein the computer program includes a plurality of instructions including one or more instructions 703 for determining at least one treatment regimen including replacement therapy for one or more steroid hormones or metabolites or modulators thereof, based on the steroid hormone levels prior to disease diagnosis in the subject and on the current steroid hormone level's in the mammalian subject. The system may further comprise one or more instructions 704 for inputting information associated with steroid hormone levels prior to disease diagnosis in a mammalian subject; one or more instructions 705 for inputting information associated with current cyclic steroid hormone levels in the mammalian subject. An output 707 and the system 702 inform a database 706 which further interacts with system 702 and output 707.

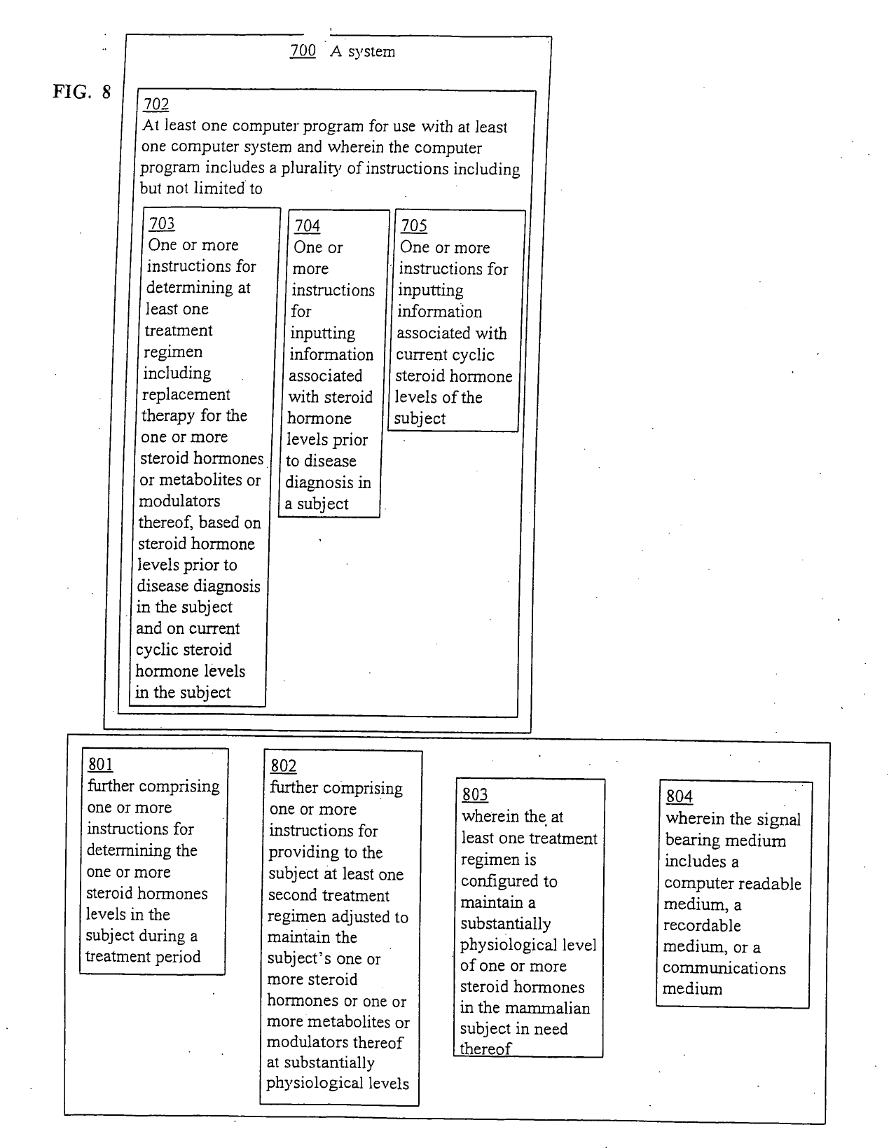

FIG. 8 depicts some aspects of a system that may serve as an illustrative environment for subject matter technologies. The system 700 as described in FIG. 7 may further comprise one or more instructions 801 for determining the one or more steroid hormones levels in the subject during a treatment period. The system 700 may further comprise one or more instructions 802 for providing to the subject at least one second treatment regimen adjusted to maintain the subject's one or more steroid hormones or one or more metabolites or modulators thereof at substantially physiological levels. In one aspect of the system 700 the at least one treatment regimen 803 is configured to maintain a substantially physiological level of one or more steroid hormones in the mammalian subject in need thereof. In a further aspect of the system 700 the signal bearing medium 804 includes a computer readable medium, a recordable medium, or a communications medium.

### Maintenance of a Substantially Physiological Level of One or More Steroid Hormones in a Mammalian Subject

### Hormone Levels in a Subject

In a method for maintaining a substantially physiological cyclic pre-menopausal level of one or more steroid hormones in a mammalian subject in need thereof, the levels of one or more steroid hormones, or metabolites or modulators thereof may be measured in one or more bodily fluids or tissues from the mammalian subject. Measurements of the levels of the one or more steroid hormones provide an individualized baseline for the substantially physiological cyclic pre-menopausal level in the subject and an indication of a need for the at least one treatment regimen including replacement therapy for the one or more steroid hormones, or metabolites or modulators thereof. Examples of bodily fluids include but are not limited to blood, serum, plasma, urine, urogenital secretions, sweat and or saliva. One or more steroid hormones or metabolites or modulators thereof that may be assayed in a bodily fluid or tissue include but are not limited to estrogen fractions such as, for example, estrone [E1], estradiol (estradiol-17β, [E2]), and estriol [E3]; progesterone; androgens such as, for example, testosterone, dihydrotestosterone (DHT), dehydroepiandrosterone (DHEA), androstenedione, androst-5-ene-3β,17β-diol; non-sterol hormones such as, for example, follicle stimulating hormone, luteinizing hormone, inhibin B, anti-Mullerian hormone, and thyroid-related hormones; and modulators, for example metabolic precursors, metabolic enzymes, and hormone receptors, such as estrogen receptor a and estrogen receptor β. One or more steroid hormones, or metabolites, modulators, or analogs thereof can be measured in one or more bodily fluids or tissues, for example, by immunoassay, gas or liquid chromatography with or without mass spectrometry, or recombinant cell based assay. The one or more steroid hormones, or metabolites, modulators, or analogs thereof can also be measured, for example, by using sensor technology, including biosensors, protein arrays, and/or microfluidic devices, which may also be referred to as “lab-on-a-chip” systems.

Levels of steroid hormones, metabolites, or modulators thereof in a subject may be assayed in a bodily fluid or tissue using an immunoassay such as, for example, an enzyme-linked immunosorbent assay (ELISA; EIA) or a radioimmunoassay (RIA). In one type of an ELISA, the analysis is based on a competitive binding reaction to a specific antibody between an analyte, e.g., the steroid hormone, in the sample and a standard, e.g. a hormone standard, which is labeled with an enzyme. The antibody itself may be immobilized on a substrate such as a microtiter plate, tube, strip or beads, for example. The amount of labeled standard bound to the immobilized antibody is inversely proportional to the amount of analyte in the sample and may be determined by the addition of a chromogenic or fluorogenic substrate, which, upon interaction with the enzyme, generates a product detectable by an instrument such as a spectrophotometer or fluorometer. In another type of ELISA, the total amount of analyte bound to the immobilized antibody is detected by a secondary antibody (which may be the same antibody as the first antibody or different) labeled with an enzyme, and the assay developed by adding a chromogenic or fluorogenic substrate. In other types of assays, the analyte, standard hormone, or secondary antibody is labeled with a tag, for example, a fluorescent tag and is detected directly. Similarly, a chemiluminescent immunoassay may be used. Alternatively, the analyte, standard hormone, or secondary antibody may labeled with ¹²⁵iodine for use in a radioimmunoassay. In these assays, quantification is determined by comparison to a standard curve generated using known amounts of analyte.

Antibodies or fragments thereof for use in an immunoassay may be generated against a hormone using standard methods, for example, such as those described by Harlow & Lane (Antibodies: A Laboratory Manual, Cold Spring Harbor Laboratory Press; 1^{st} edition 1988), which is incorporated herein by reference). Alternatively, an antibody fragment directed against a hormone may be generated using phage display technology (see, e.g., Kupper, et al. BMC Biotechnology 5:4, 2005, which is incorporated herein by reference). An antibody or fragment thereof could also be prepared using in silico design (Knappik et al., J. Mol. Biol. 296: 57-86, 2000, which is incorporated herein by reference). In addition or instead of an antibody, the assay may employ another type of recognition element, such as a receptor or ligand binding molecule. Such a recognition element may be a synthetic element like an artificial antibody or other mimetic. U.S. Pat. No. 6,255,461 (Artificial antibodies to corticosteroids prepared by molecular imprinting), U.S. Pat. No. 5,804,563 (Synthetic receptors, libraries and uses thereof), U.S. Pat. No. 6,797,522 (Synthetic receptors), U.S. U.S. Pat. No. 6,670,427 (Template-textured materials, methods for the production and use thereof), and U.S. Pat. No. 5,831,012, U.S. Patent Application 20040018508 (Surrogate antibodies and methods of preparation and use thereof); and Ye and Haupt, Anal Bioanal Chem. 378: 1887-1897, 2004; Peppas and Huang, Pharm Res. 19: 578-587 2002, provide examples of such synthetic elements and are incorporated herein by reference. In some instances, antibodies, recognition elements, or synthetic molecules that recognize a hormone may be available from a commercial source, e.g., Affibody® affinity ligands (Abcam, Inc. Cambridge, Mass. 02139-1517; U.S. Pat. No. 5,831,012, incorporated here in by reference). For example, antibodies to estradiol, estrone, estriol, testosterone, DHEA, progesterone, follicle stimulating hormone, luteinizing hormone and estrogen receptors α and β are available from numerous commercial sources as listed in the Linscott's Directory of Immunological & Biological Reagents, Linscott's USA, 6 Grove St., Mill Valley, Calif. 94941 USA. Similarly, ELISA kits designed to measure one or more hormones are commercially available. For example, ELISA kits for measuring estradiol, estrone, estriol, testosterone, DHEA, progesterone, follicle stimulating hormone, luteinizing hormone (from, e.g., Cayman Chemical, Ann Arbor, Mich.; Calbiotech, Spring Valley, Calif.; Beckman Coulter, Fullerton, Calif.). It is also anticipated that other biomolecules may be developed to selectively bind to steroid hormones or related molecules, modulators or metabolites, for example, DNA or RNA oligonucleotide based aptamers, and used in diagnostic assays (see, e.g., Jayasena. Clin. Chem. 45:1628-1650, 1999, which is incorporated herein by reference).

Alternatively, levels of one or more steroid hormones, or modulators or metabolites thereof in a subject may be assayed in a bodily fluid or tissue using gas or liquid chromatography with or without mass spectrometry. For example, estradiol and estrone levels in human plasma may be simultaneously measured using a liquid chromatography-tandem mass spectrometry assay (see, e.g. Nelson, et al., Clin. Chem. 50:373-384, 2004, which is incorporated herein by reference). In this instance, the serum samples are derivatized with dansyl chloride to increase the sensitivity of the assay and efficiency of ionization and separated from other components of the serum by liquid chromatography. Further purification and detection is done using mass spectrometry to differentiate between various steroid hormones. A more rapid method for detecting steroid hormones such as estradiol, estrone, estriol, 16-hydroxyestrone, and aldosterone, for example, using liquid chromatography, electrospray ionization and mass spectrometry (LC-ESI-MS/MS) has been described (see, e.g., Guo, et al., Clin. Biochem. 41:736-741, 2008, which is incoporated herein by reference). In this instance, the serum samples are deproteinized by extraction with acetonitrile followed by centrifugation at 13,000 rpm for 1.0 minutes. The supernatant is then loaded directly into the LC-ESI-MS/MS system where the samples are chromatographed. Standards are used to determine the elution profile of each steroid hormone and the respective peaks are submitted to electrospray ionization followed by mass spectrometry. Known quantities of a given hormone are subjected to the same process and used to generate a standard curve against which the measured levels of hormone in the serum sample are compared.

Levels of one or more steroid hormones, or modulators or metabolites thereof in a subject may also be assayed in a bodily fluid or tissue using a recombinant cell based assay or biosensor. In one instance, a yeast strain or a mammalian cell line, for example, is modified to express a recombinant hormone receptor which in response to binding an analyte, such as a steroid hormone, emits a measurable readout. For example, Klein, et al., describe development of a bioassay in Saccharomyces cerevisiae which have been transformed with the human estrogen receptor and an estrogen response element (ERE) upstream of the yeast iso-1-cytochrome C promoter fused to the structural gene for β-galactosidase (Klein, et al., J. Clin. Endocrinol. Metab. 80:2658-2660, 1995, which is incorporated herein by reference). Increased β-galactosidase activity in response to the presence of estrogen is assessed using colorimetric detection. Alternatively, a luminescent assay system or biosensor may be used to measure estrogen levels by incorporating human estrogen receptor α and/or β into a mammalian cell line in combination with an estrogen-responsive element (ERE) upstream of a luciferase gene reporter (Paris, et al., J. Clin. Endocrinol. Metab. 87: 791-797, 2002, which is incorporated herein by reference).

Levels of one or more steroid hormones, or modulators or metabolites thereof may be measured using sensor technology, including for example, chemical sensors, biosensors, protein arrays, and/or microfiuidic devices, which may also be referred to as “lab-on-a-chip” systems (see, e.g., Cheng, et al., Anal. Chem. 73: 1472-1479, 2001; Bange, et al., Biosensors Bioelectronics 20: 2488-2503, 2005; De, et al., J. Steroid Biochem. Mol. Biol. 96: 235-244, 2005; Zhou, et al., Sci. China C. Life Sci. 49: 286-292, 2006; Hansen, et al., Nano Lett., 7: 2831-2834, 2007, which are incorporated herein by reference; Dauksaite et al.; Nanotech 18(125503): 1-5, 2007). For example, a biosensor may be generated based on the interaction between estradiol and the estrogen receptor (see, e.g., Murata, et al., Anal. Sci. 17:387-390, 2001, which is incorporated herein by reference). In this instance, recombinant estrogen receptor is linked to an Au-electrode and cyclic voltametric measurements are used to assess changes in the properties of the estrogen receptor protein layer in response to estradiol binding.

In some instances, the steroid hormones, modulators, or metabolites thereof may be first extracted from the bodily fluid or tissue sample, e.g., blood, serum, plasma, urine, urogenital secretions, sweat and/or saliva, using organic solvents prior to performing one or more of the measurements described above. For example, a hormone, estradiol, may be extracted from serum using a combination of hexane and ethyl acetate followed by mixing, centrifugation, and collection of the organic layer (see, e.g., Dighe & Sluss, Clin. Chem. 50:764-6, 2004, which is incorporated herein by reference). Extracted hormones in the organic layer may be further fractionated using chromatography. For example, testosterone, dihydroestosterone, androstenedione, estrone, and estradiol extracted from serum into an organic layer may be further fractioned using Celite column partition chromatography and eluting solvents such as toluene, isooctane and ethyl acetate (see, e.g., Hsing, et al., Cancer Epidemiol. Biomarkers Prev. 16:1004-1008, 2007, which is incorporated herein by reference). Radiolabeled internal standards corresponding to a given hormone may be used to assess procedural losses.

In some instances, steroid hormone levels or modulators or metabolites thereof in a subject may be measured transdermally using a non-invasive method such as, for example, reverse ionotophoresis. In general, iontophoresis is the application of a small electric current to enhance the transport of both charged and polar, neutral compounds across the skin. Reverse iontophoresis is the term used to describe the process whereby molecules are extracted from the body to the surface of the skin in the presence of an electrical current. The negative charge of the skin at buffered pH causes it to be permselective to cations causing solvent flow towards the anode. This flow is the dominant force allowing movement of neutral molecules across the skin. This technology can be used in devices for non-invasive and continuous monitoring of compounds in interstitial fluid of individuals with disease (see, e.g., Rhee, et al., J. Korean Med. Sci. 22:70-73, 2007; Sieg, et al., Clin. Chem. 50:1383-1390, 2004; which are incorporated herein by reference)

### Time-History of Serum Hormone Levels and Dosing

Prior to determining a treatment regimen, additional information regarding the physiological status of the subject may be gathered and assessed. For example, information on the subject's own history or his or her family's history of diseases, including genetic information, may be collected. The individualized medical evaluation can include a genetic profile of the subject regarding genes, genetic mutations, or genetic polymorphisms that may indicate risk factors that affect disease related to steroid hormone levels, hormone receptors, modulators, e.g., agonists or antagonists of steroid hormones or steroid hormone receptors, or factors causing genetic disease or a genetic predisposition to disease in the subject. The individualized treatment regimen includes replacement therapy for one or more steroid hormones, or metabolites or modulators thereof. The treatment regimen can be based upon information derived from pre-menopausal hormone levels or pre-disease hormone levels in the subject. In this context, a physiological level of a hormone includes the level of hormone measured at a given time. A physiological premenopausal level can be a level of the hormone as measured at a point in time during premenopause in a female subject. A physiological pre-disease level can be a level of the hormone as measured at a point in time prior to occurrence of disease or prior to surgery to treat a disease in a female or male subject. A current physiological level can be the level of the hormone as measured just prior to determining a treatment regimen. The levels of one or more hormones, steroid hormones, modulators or metabolites thereof may be measured using the methods described herein to develop a time-history of serum hormone levels in a subject. A time-history of serum hormone levels of one or more steroid hormones, or metabolites or modulators thereof in a subject refers to the level of one or more steroid hormones, or metabolites or modulators thereof in the serum or tissue of a subject over time. As such, the level of one or more steroid hormones may be measured over any of a variety of time intervals. For example, a time-history of serum levels of one or more steroid hormone may be generated by measuring hormone levels over the course of one or more days, one or more weeks, one or more months, one or more years. In the case of a premenopausal or perimenopausal female subject, a time-history of serum levels of steroid hormones may be generated over the course of one or more menstrual cycle, for example, which may vary from 21 to 35 days. In the case of a male subject, a time-history of serum levels of steroid hormones or testosterone, for example, may be generated over the course of one or more years to assess seasonal variations in testosterone levels (see, e.g., Svartberg, et al., J. Clin. Endocrinol. Metab. 88: 3099-3104, 2003, which is incorporated herein by reference). In addition, a time-history of serum steroid hormone levels may not be contiguous in time. For example, steroid hormone levels may be measured 2-3 months out of a year, on a yearly basis, for example, and peak values or minimal values for the other months are inferred based on the average hormone levels during the measurement period. As such, a time-history of serum steroid hormone levels in a subject may be measured over multiple cycles and multiple monthly or yearly time periods.

The time-history of one or more steroid hormones, metabolites or modulators thereof in serum or tissue of a subject may be stored, analyzed and tracked. Methods for storing this information include paper storage as well as electronic storage. Analysis and tracking may be done manually by looking at the data. Ideally, a software program is designed and used to store, analyze and track the time-history of serum steroid hormones of a subject. The software program may be used to monitor changes in the time-history of serum steroid hormone levels of a subject from one measurement period to the next. The software program may compare the time-history of serum steroid hormone levels of a subject relative to steroid hormone levels associated with an age-matched population norm. The software program may also compare the steroid hormone levels of a subject to a physiological level of hormone. The physiological level of one or more steroid hormone of a subject may be inferred by measuring hormone levels at a time in the subject's life when hormone levels are assumed to be within a “normal range.” For example, in the case of a female subject, this may be during premenopause. In the case of a male subject, this may be prior to the age of 40, for example. As such, time-history of serum steroid hormones may be used to monitor changes in levels of one or more hormone relative to either a subject's own physiological level of hormone or that of a population norm. As hormone levels decline due to age, disease, or surgery, for example, supplemental hormone treatment may be used to maintain the physiological level.

The physiological cyclic level of one or more steroid hormones, metabolites or modulators thereof may be maintained by supplementing endogenous levels of steroid hormones with exogenous steroid hormones to bring the overall steroid hormone levels back to the physiological level. As such, the subject is dosed with sufficient supplemental steroid hormones, or metabolites, modulators, or analogs thereof to achieve the desired physiological level. It is anticipated that in the aging subject, the overall level of serum hormones may change over time, due, for example, to a decline in endogenous hormone even in the presence of exogenous hormone and/or to physiological changes in the subject such as a gain or loss of weight or onset of a systemic disease. As such, the hormone levels may be routinely measured, and a treatment regimen including replacement therapy for one or more steroid hormones, or metabolites or modulators thereof, adjusted appropriately to maintain the physiological steroid hormone level. The software program may be designed to include guidance regarding hormone dosing based on the measured differences in the overall hormone levels and the physiological levels.

Inferred peak values or minimal values of serum steroid hormone levels in the subject refers to steroid hormone levels in the subject that have been determined either by prior time-history of serum steroid hormone levels in the subject, a current time history, and/or by values of serum steroid hormone levels in a similar subject population determined by age, environment, family background, or genetic profile.

Treatment of Disease with Treatment Regimen Including Replacement Therapy for One or More Steroid Hormones

A treatment regimen which includes replacement therapy for one or more steroid hormones, or metabolites or modulators thereof, for use in maintaining a substantially physiological level in a subject may be used to treat a disease or symptoms associated with the loss of normal physiological hormone levels. Such a loss in hormone levels may be associated with natural or surgically induced menopause or hypogonadism, for example. In women, menopause is defined as the last menstrual cycle and is characterized by a cessation of ovarian function, leading to a significant decline in the level of circulating estrogens. The period of declining ovarian function prior to menopause is termed perimenopause and may last for several years with fluctuating estrogen levels and erratic menstrual cycles. The changes in estrogen levels during perimenopause and at menopause may cause vasomotor symptoms such as hot flashes and palpitations, psychological symptoms such as depression, anxiety, irritability, mood swings and lack of concentration, atrophic symptoms such as vaginal dryness and urgency of urination, and skeletal symptoms such as osteopenia and muscle pain. Menopause may be induced artificially by surgical removal of the ovaries. The symptoms associated with perimenopause, menopause, and post-menopause may be treated with estrogens either with or without progestin. Progestin is added to the treatment regime, for example, to prevent estrogen-induced endometrial proliferation and cancer in women with intact uteri.

Aging men also exhibit a natural decline in steroid hormones including, for example, decreased testosterone, estrone, androstanediol glucuronide, dehydroepiandrosterone, and dehydroepiandrosterone sulfate. For example, the normal levels of testosterone range from 270 to 1000 nanograms/deciliter in men under 40 but begin to decline on average 0.8%/year after the age of 40 (see, e.g., Feldman, et al., J. Clin. Endocrinol. Metab. 87:589-598, 2002, which is incorporated herein by reference). The decline in testosterone in aging men has been associated with parallel age declines in bone mass, muscle mass/strength, physical function/frailty, and sexual function with symptoms ranging from irritability, nervousness, anxiety, sweating, sleep disturbances, decreased energy, decreased beard growth, and decreased potency, morning erections, and libido. In addition, the reduction in testosterone may be linked with various age-associated metabolic changes such as abdominal obesity, diabetes, and markers of prediabetes (see, e.g, Araujo, et al., J. Clin. Endocrinol. Metab. 92:4241-4247, 2007, which is incorporated herein by reference). Testosterone levels may also decline or be absent all together (hypogonadism) for reasons other than aging. For example, hypogonadism in man may be due to problems with the testes themselves or the pituitary gland. This includes disorders of the testes such as Klinefelter's syndrome, inflammation of the testes (orchitis), radiation or chemotherapy, and alcohol abuse. Removal of both testicles, injury to both testicles and undescended testicles are all causes of hypogonadism. Any disease of the pituitary gland may also result in hypogonadism. As such, supplemental testosterone therapy in the form of transdermal patches, gels and creams, for example, may be used to relieve the symptoms associated with the decline or lack of testosterone (see, e.g., Bain Canadian Family Physician 47:91-97, 2001, which is incorporated herein by reference).

In addition to relieving the symptoms associated with age-, disease- or surgery-related decrease in one or more hormones, maintaining a substantially physiological level of one or more steroid hormones, may be of use in preventing or slowing the onset or progression of a disease such as for example bone degeneration, neurological disease, cancer, metabolic disease, and cardiovascular disease.

A treatment regimen to maintain a substantially physiological level of one or more steroid hormones, or metabolites or modulators thereof, may have benefit in slowing the loss in bone mineral density associated with age and declining hormone levels. In women, bone mineral density has been correlated with age of menarche, parity, age of menopause, and cumulative lifetime exposure to endogenous and exogenous estrogens (see, e.g., Tan, et al., Arch. Neurol. 62:107-111, 2005, which is incorporated herein by reference). Estrogen replacement has shown benefit in preventing the loss of bone mineral density and in preventing fractures. For example, the daily use of estrogen-plus-progestin therapy (Prempro; 0.625 mg conjugated equine estrogen/2.5 mg medroxyprogesterone acetate) for 6 months or more in a multicenter, placebo-controlled clinical trial with over 16,000 participants resulted in a statistically significant decrease in osteoporotic fractures (8.6% treated versus 11.1% placebo; hazard ratio=0.79; 95% confidence interval 0.68-0.83; see Cauley, et al., JAMA 290:1729-1738, 2003, which is incorporated herein by reference). In this same study, bone mineral density increased 3.7% after 3 years of estrogen-progestin treatment. Similarly, both testosterone and dehydroepiandrosterone (DHEA) treatments have been shown to improve hip and spine bone mineral density, although the use of testosterone for this purpose in men is limited by the potential risk of prostate cancer and has not been extensively studied in women (see, e.g., Suzuki, et al. J. Pharmacol. Sci. 106:530-535, 2008, which is incorporated herein by reference).

As another example, a treatment regimen to maintain physiological cyclic levels of steroid hormones, or modulators or metabolites thereof, may prevent loss of cognitive function and protect against mild cognitive impairment, dementia, and neurodegenerative diseases such as, for example, Alzheimer's disease, Parkinson's disease and amyotrophic lateral sclerosis (ALS). For example, estrogens have been shown in vitro to decrease the accumulation of neurotoxic glutamate and β-amyloid peptide, inhibit neuronal apoptosis, act as an anti-oxidant, and modulate the gene expression of apolipoprotein E, and as such may have a global effect on cognitive function (see, e.g., Morrison, et al., J. Neurosci. 26:10332-10348, 2006; Ryan, et al., Int. Psychogeriatr. 20:47-56, 2008, which are incorporated herein by reference). In vivo, lifetime exposure to endogenous estrogen may affect cognitive decline. For example, in a cohort of women who had never received estrogen replacement therapy, cognitive decline was less in those women who had had no children (nulliparity) and/or had reached menopause at a later age (see, e.g., McLay, et al., J. Neuropsychiatry Lin. Neurosci. 15:161-167, 2003, which is incorporated herein by reference). Similarly, administration of exogenous estrogen may affect cognitive decline. For example, recent meta-analyses regarding estrogen treatment and Alzheimer's suggest a possible reduction in disease risk of 29 to 44% (see, e.g., Ryan, et al. Int. Psychogeriatr. 20:47-56, 2008, which is incorporated herein by reference). The Women's Health Initiative Memory Study (WHIMS) assessed the effect of daily estrogen/progestin treatment on memory in women 65 years and older and found an increased risk of developing dementia and preventative effect on mild cognitive impairment (see Schuniaker, et al., JAMA, 289:2651-2662, 2003, which is incorporated herein by reference). However, a number of additional analyses suggest that initiating estrogen replacement earlier during perimenopause, for example, and using natural estrogen versus conjugated equine estrogen may provide increased protection against mild cognitive impairment and Alzheimer's disease (see, e.g., Ryan, et al. Int. Psychogeriatr. 20:47-56, 2008; Morrison, et al., J. Neurosci. 26:10332-10348, 2006, which are incorporated herein by reference). Studies in animals also support the importance of uninterrupted exposure to hormones for neuroprotective and anti-inflammatory efficacy (see, for example, Suzuki et al. PNAS USA, 104: 6013-6018, 2007, which is incorporated herein by reference). Reduced cognitive ability has also been linked with low endogenous levels of testosterone in aging men, and as such testosterone substitution may improve some aspects of cognitive ability (see, e.g., Beauchet, Eur. J. Endocrinol. 155:773-781, 2006, which is incorporated herein by reference).

A treatment regimen to maintain physiological cyclic levels of one or more steroid hormones, or modulators or metabolites thereof, may have benefit in reducing the risk of developing certain types of cancer. For example, in a large randomized controlled study, postmenopausal women who were treated on average for 5 years with a combination of estrogen and progestin had a reduced risk of developing colorectal cancer relative to women treated with a placebo with a hazard ratio of 0.63 (nominal 95% confidence intervals of 0.43-0.92; see, e.g., Writing Group for the Women's Health Initiative Investigators. JAMA 288: 321-333, 2002, which is incorporated herein by reference). In this same study, the hazard ratio for the development of endometrial cancer was 0.83 (0.47-1.47) in the estrogen/progestin-treated women relative to those treated with placebo, suggesting a possible protective effect of hormone therapy against development of endometrial cancer, as well as colorectal cancer. There is also evidence to suggest that hormone therapy may lower the risk for developing non-small cell lung cancer (NSCLC). In one study, for example, a treatment regimen including replacement therapy for one or more steroid hormones or metabolites or modulators thereof reduced the risk of developing lung cancer by 33% overall (hazard ratio=0.67; 95% confidence interval=0.53-0.85; see, e.g., Ramnath, et al., Oncology 73:305-310, 2007, which is incorporated herein by reference). The hazard ratio in former smokers who used hormone therapy relative to non-users was even lower at 0.55 (0.41-0.75). In another study, a treatment regimen that included replacement therapy for steroid hormones or metabolites or modulators thereof reduced the risk of developing cancer of the large bowel or rectum, uterine body, or ovaries. Hannaford et al., BMJ, 335: 651-658, 2007; Booth et al., Am J Physiol Heart Circ Physiol, 293: H1408-H1415, 2007, which are incorporated herein by reference.

A treatment regimen to maintain substantially physiological cyclic levels of one or more steroid hormones, or metabolites or modulators thereof, may have protective benefits in cardiovascular disease. For example, a treatment regimen determined based on a subject's physiological pre-menopausal hormone levels and current physiological hormone levels may include providing an amount and type of estrogen, administered in a particular fashion, to a woman whose estrogen levels only recently decreased, such as a woman who is transitioning into menopause or in early menopause, or a woman has recently lost ovary function due to surgery, exposure, or disease. In animals and humans endogenous and exogenously provided estrogen is protective against atherogenesis and cardiovascular disease in general, especially in younger women. For example, later age of menopause and longer exposure to endogenous estrogens is associated with protection against cardiovascular disease (see van der Schouw et al., Lancet 16:714-8 1996, which is incorporated herein by reference), and premature atherosclerosis common in women and primates with premenopausal estrogen deficiency can be prevented by estrogen treatment (see Clarkson, Menopause 14: 373-84, 2007, which is incorporated herein by reference). Studies in animals also demonstrated that the timing of initiation of estrogen treatment (e.g., 17β-estradiol, E2) after loss of ovarian hormone function in a subject is a major indicator for successful therapeutic cardiovascular outcomes (Pinna et al., Hypertension 51: 1210-1217, 2008). In the estrogen-only arm of the WHI trial, an analysis of the 50-59-year-old age group showed a near statistical decrease in coronary events: 63 (0.36-1.08), and a statistically significant reduction in a global coronary score, 0.66 (0.45-0.96) (see Hsia, et al., Arch Intern. Med. 2006; 166:357-365). Current therapy and Clinical Trials (see, e.g., The Kronos Early Estrogen Prevention Study (KEEPS)) are now focusing on treating women transitioning into menopause and early menopause with estrogens alone or in combination administered orally or transdermally (Harman, et al., Climacteric 8(1):3-12, 2005; Miller et al., J. Appl. Physiol. 99: 381-383, 2005; which are incorporated herein by reference.). Women, for example young women with estrogen deficiency due to oophorectomy, disease or early menopause, as well as women transitioning through menopause may benefit in protection against cardiovascular disease by a method to maintain a physiological level of one or more steroid hormones that includes providing a treatment regimen including replacement therapy for one or more steroid hormones or metabolites or modulators thereof. The treatment regimen includes selecting a single specific form of estrogen, e.g., a natural estradiol, and/or a particular means of delivery, e.g., transdermal delivery, and/or including providing an antagonistic tissue-specific estrogen receptor modulator (SERM) to inhibit responses in certain tissues. (Qiao, et al. in Gender Medicine. 5 Suppl. A, S46-S64, 2008, which is incorporated herein by reference.) In another example, progesterone therapy, for example as part of a treatment regimen including replacement therapy for one or more steroid hormones or modulators or metabolites thereof, may be useful for treatment of inflammatory disorders and as a cardioprotective agent against reperfusion injury resulting from a myocardial ischemia. See, e.g. Booth et al., Am J Physiol Heart Circ Physiol, 293: H1408-H1415, 2007; Booth et al., J Pharmacol Exp Ther, 307: 395-401, 2003, which are incorporated herein by reference.

A treatment regimen to maintain physiological cyclic levels of steroid hormones or metabolites or modulators thereof in a subject may have benefit in metabolic disease. For example, treatment of postmenopausal women with estrogen and progestin for the relief of menopausal symptoms is associated with improved glycemic control in those women who also have Type 2 diabetes (see, e.g., Ferrara, et al., Diabetes Care 24:1144-1150, 2001, which is incorporated herein by reference). Women with diabetes who received hormone replacement therapy had a significant decrease in glycosylated hemoglobin, an indirect measure of glucose levels. Similarly, estrogen alone or in combination with progestin improves lipoprotein accumulations and lowers fibrinogen levels without detectable effects on postchallenge insulin or blood pressure (see, e.g., The Writing Group for the PEPI Trial, JAMA, 273: 199-208, 1995, which is incorporated herein by reference).

### Genetic Profiling

Prior to determining a treatment regimen, additional information regarding the physiological status of the subject may be gathered and assessed. For example, information on the subject's own history or his or her family's history of diseases, including genetic information, may be collected. The medical evaluation can include a genetic profile of the subject regarding genes, genetic mutations, or genetic polymorphisms that may indicate risk factors that affect disease and/or are related to steroid hormone levels, hormone receptors, modulators, e.g., agonists or antagonists, of steroid hormones or steroid hormone receptors, or factors causing genetic disease or a genetic predisposition to disease in the subject. Medical evaluation regarding genetic profiling or genetic testing can be provided as a current determination of genetic risk factors, or as part of the subject's medical history. Genetic profiling or genetic testing can be used to design a treatment regimen and determine an optimal level individualized for the subject of one or more steroid hormones, steroid hormone receptors, metabolites, or Modulators thereof, wherein the genetic profile was obtained during a pre-menopausal or pre-disease period from the subject. A physician may use the genetic profiling or genetic testing information to determine a genetic basis for needed treatment to maintain a substantially physiological cyclic level of one or more steroid hormones in a mammalian subject in need thereof. Determining a genetic profile of a subject may be used to predict the potential response to a treatment regimen designed to maintain physiological cyclic levels of one or more steroid hormones, or modulators or metabolites thereof. In addition, genetic profiling, of a subject may predict the risk of developing a chronic or life threatening disease that may be attenuated or prevented by providing a treatment regimen including one or more steroid hormones, or metabolites or modulators, or analogs thereof. In general, genetic profiling refers to analysis of a subject's genomic DNA for the purpose of comparing with known genetic information.

A genetic polymorphism or genetic mutation in a genetic profile of a subject that encodes a component of one or more hormone signaling pathway may affect the levels of the levels of hormones and related compounds. As such, genetic profiling may be used prior to the initiation of a treatment regimen including providing one or more steroid hormones, or metabolites, modulators, or analogs thereof; to assess whether the subject has any genetic mutations and/or genetic polymorphisms that may be historically correlated with levels of one or more steroid hormones, or metabolites, modulators, or analogs thereof. For example, the enzyme CYP17 mediates both steroid 17a-hydroxylase and 17,20-lyase activities and is essential for the production of steroid hormones including estrogen and testosterone. Polymorphisms in this gene have been associated with altered estrogen levels, and may have additional effects on hormone related diseases (Jasienka, et al., Cancer Epidemiology, Biomarkers and Prevention 15: 2131-2135, 2006; Small, et al., Human Reproduction 20(8): 2162-2167, 2005; and Sharp et al., Am J Epidemiol 160: 729-740, 2004; which are incorporated herein by reference). In one example, women homozygous for the CYP17 A2 allele polymorphism have increased levels of estradiol during each menstrual cycle and during menopause are half as likely to need hormone replacement therapy for menopausal symptoms (Feigelson, et al., Canc. Res. 59:3908-3910, 1999, which is incorporated herein by reference). The complete genomic DNA sequence as well as the coding DNA sequence for this enzyme and other components of pathways associated with steroid hormones can be found, for example, in the National Center for Biotechnology Information (NCBI) database (see, e.g., http://www.ncbi.nlm.nih.gov/).

A polymorphism or mutation in the genetic information of a subject that encodes a component of one or more hormone signaling pathway may dictate how well that subject will respond to treatment with one or more steroid hormones, or metabolites, modulators, or analogs thereof. As such, genetic profiling may be used prior to the initiation of a treatment regimen including one or more steroid hormones, or metabolites, modulators, or analogs thereof to assess whether the subject has any genetic mutations and/or polymorphisms that may be historically correlated with a positive or negative response to a treatment regimen to maintain physiological cyclic levels of one or more steroid hormones, or metabolites, or modulators thereof in the subject. Of particular interest are potential mutations or polymorphisms associated with either hormone receptors or other components of the hormone signaling pathway that may differentially respond to supplemental hormone treatment. Receptors that would be of interest for genetic profiling include but are not limited to the estrogen receptors (ERα and ERβ), the androgen receptor (also known as NR3C4, nuclear receptor subfamily 3, group C, member 4), the progesterone receptor (also known as NR3C3, nuclear receptor subfamily 3, group C, member 4), the follicle stimulating hormone receptor (FSHr), luteinizing hormone receptor (LHr), and anti-Mullerian receptor type II. The complete genomic DNA sequence as well as the coding DNA sequence for these and other relevant targets can be found, for example, in the National Center for Biotechnology Information (NCBI) database (http://www.ncbi.nlm.nih.gov/).

A number of polymorphisms have been studied in association with the estrogen receptor. For example, a single nucleotide polymorphism in the estrogen receptor a gene confers positive changes in lumbar bone mineral density following hormone replacement therapy (see, e.g., Yahata, et al., Hum. Reprod. 20:1860-1866, 2005, which is incorporated herein by reference). In this instance, women with the non-coding genotype IVS6+141441 showed significant increases in bone mineral density ranging on average from 5.0 to 8.0% in each year of three years of hormone replacement therapy relative to women lacking the IVS6+141441 genotype. Polymorphisms in the estrogen receptor may also confer positive changes in HDL cholesterol levels in response to hormone replacement therapy (see, e.g., U.S. Pat. No. 6,828,103, which is incorporated herein by reference).

Medical evaluation of the subject for genetic profiling or genetic testing to determine gene polymorphisms may be provided as a current determination of genetic risk factors in the subject, or as part of the subject's medical history. In some instances, polymorphisms in the estrogen receptor or other hormone receptor may be associated with an increased risk of developing a specific disease, which may inform a physician as to whether or not hormone treatment would be appropriate for a particular subject. For example, polymorphisms in estrogen receptor β gene variants are associated with increased risk of Alzheimer's disease in women (see, e.g., Priskanen, et al., Eur. J. Hum. Genet. 13:1000-1006, 2005, which is incorporated herein by reference). Similarly, the severity of cardiovascular disease in both men and women may be correlated with polymorphisms in the estrogen receptor a gene. For example, severity of coronary artery disease in postmenopausal women, as judged by the number of vessels with 50% stenosis, was greater in women carrying the PvuII CT genotype and the XhaI GA genotype relative to the other PvuII and XbaI genotypes (see, e.g., Alevizaki, et al., Eur. J. Endocrinol. 156:489-496, 2007, which is incorporated herein by reference).

Polymorphisms in a hormone receptor gene may predict relative response to a given hormone in terms of how well the receptor uses available hormone and how well the resulting signaling events are transmitted. For example, the PROGINS polymorphisms in the human progesterone receptor reduce the stability of the receptor mRNA transcript, reduce transactivation activity of the receptor, and reduce the efficiency of progestin-induced inhibition of cell proliferation (see, e.g., Romano, et al., J. Mol. Endocrinol. 38:331-350, 2007, which is incorporated herein by reference). Women who carry the PROGRINS polymorphisms are at increased risk for developing ovarian cancer, endometrial cancer and endometriosis. In another example, polymorphisms in the follicle stimulating hormone receptor resulting in a single amino acid change from asparagine to serine results in lower sensitivity to FSH, decreased negative feedback and longer menstrual cycles (see, e.g., Greb, et al., J. Clin. Endocrinol. Metab. 90:4866-4872, 2005, which is incorporated herein by reference).

Genetic profiling of potential disease markers or risk indicators may be used as part of treating a subject with one or more steroid hormones, or metabolites, modulators, or related compounds to maintain a physiological level of the steroid hormones. Genetic profiling may be used to assess whether or not a subject is at risk for developing a chronic or life threatening disease that might be attenuated or prevented by use of supplemental hormone treatment. Chronic or life threatening diseases of interest include but are not limited to bone degeneration, neurological disease, cancer, metabolic disease, and cardiovascular disease. For example, development of late-onset Alzheimer's disease is associated with a specific polymorphism in apolipoprotein E (APOE) termed the ε4 genotype (see, e.g., Strittmatter, et al., Proc. Natl. Acad. Sci., USA., 90:1977-1981, 1993, which is incorporated herein by reference). As another example, a polymorphism in the NEDD9 gene (neural precursor cell expressed, developmentally down-regulated gene) has been correlated with an increased risk of developing late-onset Alzheimer's disease and Parkinson's disease with odd ratios of 1.38 (1.20-1.59) and 1.31 (1.05-1.62), respectively (see, Yonghong, et al., Hum. Mol. Genet. 17:759-767, 2008, which is incorporated herein by reference).

Genetic profiling can be used to identify subjects with a predisposition to hypertension and cardiovascular disease. For subjects determined to have a predisposition to hypertension and cardiovascular disease, a treatment regimen including replacement therapy for one or more steroid hormones or metabolites or modulators thereof, can be developed to reduce the incidence of hypertension and cardiovascular disease in female subjects. These diseases may develop during transitions from premenopause to perimenopause, early menopause, late menopause, and/or post menopause. Qiao et al., Gender Medicine 5: Suppl. A, S46-S64, 2008, which is incorporated herein by reference.

Polymorphisms or mutations in a number of genes have been linked to increased risk of developing osteoporosis. These include but are not limited to lipoprotein receptor-related protein 5 (LRP5), transforming growth factor β1 (TGF-β1), bone morphogenic proteins (BMPs), sclerostin, CBFA1 gene, cathespin K, TCIRG1 gene, CLCN7 gene, Vitamin D receptor, collagen types Iα I (COLIA1), and estrogen receptor α (see, e.g., Ralston and deCrombrugghe, Genes & Dev. 20:2492-2506, 2006, which is incorporated herein by reference). For example, amino acid substitutions Ala1330Val and Val667Met in LRP5 increase the risk of developing osteoporosis in older men with odds ratios ranging from 1.01 to 8.81 (see, e.g., Brixen et al, Calcif: Tissue Int. 81:421-429, 2007, which is incorporated herein by reference).

As discussed above, a treatment regimen to maintain physiological cyclic levels of steroid hormones, or metabolites thereof may have benefit in reducing the risk of developing certain types of cancer, e.g., colorectal cancer, endometrial cancer, large bowel or rectal cancer, uterine cancer, ovarian cancer, or non-small cell lung cancer. Genetic profiling can identify subjects at risk for developing certain types of cancer, including those related to steroid hormones and related pathways. Genetic profiling can identify polymorphisms in specific genes that have been linked with increased risk of developing specific cancers. For example, polymorphisms represented by single amino acid substitutions in several DNA repair genes including xeroderma pigmentosum complementation group D (XPD), xeroderma pigmentosum complementation group F (XPF), X-ray repair cross-complementing group 1 (XRCC1), and X-ray repair cross-complementing group 3 (XRCC3) have been associated with an increased risk of developing non-small cell lung cancer (see, e.g., Butkiewicz, et al., Carcinogenesis. 22:593-597, 2001, which is incorporated herein by reference). For example, with the XPD Asp312Asn polymorphism, the Asp/Asp genotype is associated with a risk of developing lung cancer as an odds ratio of 1:39 relative to the Asn/Asn genotype. The odds ratio in individuals with the Asp/Asp genotype who are also smokers jumps to 5.32 (0.35-21.02). Other examples of polymorphisms linked to increased disease risk may be obtained from the medical literature.

Genomic DNA for use in genetic profiling may be isolated from any biological sample which contains the DNA of that subject including but not limited to blood, saliva, cheek swab, or tissue. For example, genomic DNA may be extracted from whole blood or from isolated peripheral blood leukocytes isolated by differential centrifugation from whole blood using a commercially available DNA purification kit (see, e.g., QIAamp DNA Blood Mini kit, Qiagen, Valencia, Calif.) using the manufacturer's instructions.

Medical evaluation of the subject for genetic profiling or genetic testing may be provided as a current determination of genetic risk factors in the subject, or as part of the subject's medical history. Genetic profiling or genetic testing may be carried out using a variety of methods including but not limited to restriction landmark genomic scanning (RLGS), southern blot analysis combined with restriction fragment length polymorphism (RFLP), fluorescence in situ hybridization (FISH), enzyme mismatch cleavage (EMC) of nucleic acid heteroduplexes, ligase chain reaction (LCR), and polymerase chain reaction (PCR) based methods (Tawata, et al., Comb. Chem. High Throughput Screen. 3:1-9, 2000, which is incorporated herein by reference). Analysis of one or more single nucleotide polymorphisms (SNPs) may also be used for genetic profiling.

Restriction fragment landmark genomic scanning (RLGS) may be used to scan an entire mammalian genome. As such, genomic DNA is digested with restriction enzymes to generate large DNA fragments. The fragments are separated on an agarose gel, digested with one or more restriction enzymes within the agarose gel, and then separated in a second dimension by polyacrylamide gel electrophoresis (PAGE) (Tawata, et al., Comb. Chem. High Throughput Screen. 3:1-9, 2000, which is incorporated herein by reference). The DNA may be labeled prior to digestion, or the fragments may be stained nonspecifically as with an intercalating dye, for example. The resulting pattern may be compared with pre-established norms to detect genetic mutations.

Restriction fragment length polymorphism (RFLP) is similar to restriction fragment landmark genomic scanning in that the genomic DNA is digested with specific restriction enzymes and separated on an agarose gel. The separated DNA is transferred to a membrane and the fragments are visualized using hybridization analysis and gene specific probes.

A variety of PCR related methods may be used for genetic profiling and may be used to detect both known and unknown mutations and polymorphisms (Tawata, et al., Comb. Chem. High Throughput Screen. 3:1-9, 2000, which is incorporated herein by reference). For known mutations and polymorphisms, specific PCR oligonucleotide probes are designed to bind directly to the mutation or polymorphism or proximal to the mutation or polymorphism. For example, PCR may be used in combination with RFLP. In this instance, a DNA fragment or fragments generated by PCR with primers on either side of the mutation or polymorphism site are treated with restriction enzymes and separated by agarose gel electrophoresis. The fragments themselves may be detected using an intercalating dye such as, for example, ethidium bromide. An aberrant banding pattern may be observed if mutations exist within the restriction sites. PAGE may be used to detect single base differences in the size of a fragment.

Alternatively, PCR may be used in combination with DNA sequencing for genetic profiling. For example, PCR primers may be designed that bind to either side of a potential mutation site on the target DNA and generate a PCR fragment that spans a potential mutation site. The PCR fragment is either directly sequenced or subcloned into a cloning vector and subsequently sequenced using standard molecular biology techniques.

Alternatively, a mutation or polymorphism may be screened using comparative genomic hybridization (CGH) (Pinkel & Albertson, Nature Gen. 37:S11-S17, 2005, which is incorporated herein by reference). In this instance, “normal” genomic DNA and test genomic DNA are differentially labeled and hybridized to metaphase chromosomes or DNA microarrays. The relative hybridization signal at a given location is proportional to the relative copy number of the sequences in the reference and test genomes. Arrays may be generated using DNA obtained from, for example, bacterial artificial chromosomes (BACs) or PCR.

Analysis of one or more single nucleotide polymorphism (SNP) may be used for genetic profiling. A SNP is a DNA sequence variation in which a single nucleotide in the genomic sequence differs between members of a species (or between paired chromosomes of an individual). For a variation to be considered a SNP it must occur in at least 1% of the population. Most SNPs do not affect protein function, and/or are not responsible for a disease state, but they may serve as biological markers for pinpointing an altered protein or disease on the human genome map as they are often located near a gene found to be associated with a certain disease. Occasionally, a SNP may actually affect protein function and/or cause a disease and, therefore, can be used to search for and isolate a specific gene, e.g., a T to C mutation in the CYP17 gene which affects enzyme function. The pattern of SNPs in a subject's genomic DNA may be compared with information in databases in an association study to determine effect on protein function and/or risk of disease development. SNPs may be identified using PCR and DNA sequencing as described above. Alternatively, SNP genotyping may be done using high throughput array analysis (see, e.g., Applied BioSystems, ABI PRISM, 3100 Genetic Analyzer with 22-cm Capillary Array; Syvanen, et al., Nature Genetics, 37: S5-S10, 2005, which is incorporated herein by reference). A growing number of web-based databases are available for finding information regarding SNPs and protein function and/o disease associations (see, e.g., International HapMap Project: http://snp.cshl.org/; Nature 449: 851-861, 2007; National Center Biotechnology Information (NCBI) Single Nucleotide Polymorphisms, http://www.ncbi.nlm.nih.gov/projects/SNP/)

### Drug Delivery/Time Release Device

A treatment regimen which includes one or more steroid hormones, or metabolites, modulators, or analogs thereof for use in maintaining a substantially physiological level in a subject may be administered to a mammalian subject by a variety of methods such as, for example, via oral, parenteral, subcutaneous, intravenous, intramuscular, intraperitoneal, transdermal, transbuccal, intraocular, or intravaginal routes, e.g., by inhalation, intra-nasal spray, by depot injections, or by hormone implants.

Pharmaceutical compositions containing one or more steroid hormones or metabolites, modulators, or analogs thereof and a suitable carrier can be solid dosage forms which include, but are not limited to, tablets, capsules, cachets, pellets, pills, powders and granules; topical dosage forms which include, but are not limited to, solutions, powders, fluid emulsions, fluid suspensions, semi-solids, ointments, pastes, creams, gels and jellies, and foams; and parenteral dosage forms which include, but are not limited to, solutions, suspensions, emulsions, and dry powders.

The administration of one or more steroid hormones, or metabolites, modulators, or analogs thereof to a mammalian subject may constitute a single dose, multiple daily doses, multiple doses per day, continuous infusion and or time released dose. A cyclic, continuous or combination dosing regime may be used. For example, estrogen may be taken for 25 days each month with progestin added for 10 to 12 days, and no medication used for 3 to 6 days per month. Menstrual-like bleeding is expected during the period when no medication is taken in women who have not reached menopause. Alternatively, estrogen may be given daily with progestin added for 10 to 14 days per month. Alternatively, estrogen and progestin may be given continuously as a daily oral tablet, for example.

One or more steroid hormones, or metabolites, modulators, or analogs thereof may be administered orally using, for example, push-fit capsules made of gelatin or soft sealed capsules made of gelatin and a plasticizer such as glycerol or sorbitol. One or more hormone may be combined with fillers such as, e.g., lactose, binders such as, e.g., starches, and/or lubricants such as, e.g., talc or magnesium stearate and, optionally, stabilizers. In soft capsules, one or more hormone may be dissolved or suspended in suitable liquids, such as fatty oils, liquid paraffin, or liquid polyethylene glycols. In addition, stabilizers can be added.

One or more steroid hormones, or metabolites, modulators, or analogs thereof may be administered by inhalation using an aerosol spray from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount.

One or more steroid hormones, or metabolites, modulators, or analogs thereof may be formulated for parenteral administration by injection, e.g., by bolus injection or continuous infusion. One or more steroid hormones may be administered by continuous infusion subcutaneously over a period of about 15 minutes to about 24 hours. In some instances, continuous infusion may be done over the course of days and/or months. Compositions for injection can be presented in unit dosage form, e.g., in ampoules or in multi-dose containers, with an added preservative. The compositions can take such forms as suspensions, solutions or emulsions in oily or aqueous vehicles, and can contain agents such as suspending, stabilizing and/or dispersing agents.

### Transdermal Delivery Method

In general, a treatment regimen to maintain physiological cyclic levels of one or more steroid hormones or metabolites or modulators thereof, may be delivered through or across the skin of a subject using either passive or active transdermal delivery methods. Passive transdermal delivery methods utilize passive diffusion of agents across the skin and are exemplified by adhesive transdermal patches. In this instance, a patch is applied to the skin of a subject and one or more steroid hormones slowly and continuously diffuses out of the patch at a rate dictated by the formulation of one or more hormone and the composition of the patch. For example, transdermal administration of estrogen is known in the art and described in U.S. Pat. Nos. 4,460,372; 4,573,996; 4,624,665; 4,722,941; and 5,223,261; which are incorporated herein by reference.

A transdermal patch for administering one or more steroid hormones, or metabolites, modulators, or analogs thereof may include a non-permeable backing layer, a permeable surface layer, an adhesive layer, and a reservoir containing hormone as described in U.S. Patent Publication 2008/0119449, which is incorporated herein by reference.

Examples of suitable materials which may comprise the non-permeable backing layer are well known in the art of transdermal patch delivery and include but are not limited to polyester film, such as high density polyethylene, low density polyethylene or composites of polyethylene; polypropylene; polyvinyl chloride, polyvinylidene chloride; ethylene-vinyl acetate copolymers; and the like.

Examples of suitable permeable surface layer materials are also well known in the art of transdermal patch delivery, and any conventional material which is permeable to the one or more hormone to be administered, may be employed. Specific examples of suitable materials for the permeable surface layer include but are not limited to dense or microporous polymer films such as those comprised of polycarbonates, polyvinyl chlorides, polyamides, modacrylic copolymers, polysulfones, halogenated polymers, polychloroethers, acetal polymers, acrylic resins, and the like (see, e.g., U.S. Patent Publication 2008/0119449, which is incorporated herein by reference).

Examples of suitable adhesives which may be coated on the backing layer to provide the adhesive layer are also well known in the art and include, for example pressure sensitive adhesives such as those comprising acrylic and/or methacrylic polymers. Specific examples of suitable adhesives include polymers of esters of acrylic or methacrylic acid (e.g., n-butanol, n-pentanol, isopentanol, 2-methyl butanol, 1-methyl butanol, 1-methyl pentanol, 3-methyl pentanol, 3-methyl pentanol, 3-ethyl butanol, isooctanol, n-decanol, or n-dodecanol esters thereof) alone or copolymerized with ethylenically unsaturated monomers such as acrylic acid, methacrylic acid, acrylamide, methacrylamide, N-alkoxymethyl acrylamides, N-alkoxymethyl methacrylamides, N-t-butylacrylamide, itaconic acid, vinyl acetate, N-branched C.sub.10-24 alkyl maleamic acids, glycol diacrylate, or mixtures of the foregoing; natural or synthetic rubbers such as silicon rubber, styrene-butadiene rubber, butyl-ether rubber, neoprene rubber, nitrile rubber, polyisobutylene, polybutadiene, and polyisoprene; polyurethane elastomers; vinyl polymers such as polyvinyl alcohol, polyvinyl ethers, polyvinyl pyrrolidone, and polyvinyl acetate; ureaformaldehyde resins; phenol formaldehyde resins; resorcinol formaldehyde resins; cellulose derivatives such as ethyl cellulose, methyl cellulose, nitrocellulose, cellulose acetatebutyrate, and carboxymethyl cellulose, and natural gums such as guar, acacia, pectin, starch, destria, gelatin, casein, and the like.

One or more steroid hormones, or metabolites, modulators, or analogs thereof may be administered by active transdermal delivery methods which utilize an energy source to increase the flux of the one or more hormone across the skin either by altering the barrier function of the skin (primarily the stratum corneum) or by increasing the energy of the hormone molecules. In this instance, the level of one or more steroid hormones delivered through the skin to the subject may be proportional to the overall level of energy applied.

Energy sources for use in active transdermal delivery include, but are not limited to, electrical (e.g., iontophoresis and electroporation), ultrasonic (phonophoresis, sonophoresis), magnetic (magnetophoresis), and thermal energies (see, e.g., Gordon, et al., “Transdermal Delivery: 4 Myths about transdermal drug deliver”, Drug Delivery Technology, 3(4): June 2003 which is incorporated herein by reference). Iontophoresis, for example, uses low voltage electrical current to drive ionized agents or drugs across the skin. An electric current flows from an anode to a cathode, with the skin completing the circuit and drives ionized molecules into the skin from a reservoir associated with the transdermal delivery device. By contrast, electroporation uses short electrical pulses of high voltage to create transient aqueous pores in the skin through which an agent or drug may be transported. Phonophoresis or sonophoresis uses low frequency ultrasonic energy to disrupt the stratum corneum. For example, Saliba et al. describe enhanced systemic levels of topical dexamethasone when applied in combination with ultrasound pulsed with an intensity of 1.0 W/cm² at a frequency of 3-MHz for 5 minutes (Saliba, et al., J. Athletic Training. 43:349-354, 2007, which is incorporated herein by reference). Thermal energy may be used to facilitate transdermal delivery by making the skin more permeable and by increasing the energy of drug molecules. In addition, one or more chemical permeation enhancer may be included. Examples of such enhancers include, but are not limited, to isopropyl myristate, bile salts, surfactants, fatty acids and derivatives, chelators, cyclodextrins or chitosan.

In some instances, transdermal delivery of one or more steroid hormones or metabolites, modulators, or analogs thereof may be faciliated using microporation induced by an array of microneedles. Microneedles, when applied to the skin, painlessly create micropores in the stratum corneum without causing bleeding and lower the resistance to drug diffusion through the skin. The microneedles may be used to abrade or ablate the skin prior to transdermal transport of one or more hormone. For example, a micro-array of heated hollow posts may be used to thermally ablate human skin in preparation for transdermal drug delivery by diffusion as described in U.S. Patent Application 2008/0045879, which is incorporated herein by reference. Alternatively, an array of microfine lance's or microneedles may be designed to actively inject drug into the skin as described in Roxhed, et al., IEEE Transactions on Biomedical Engineering, 55:1063-1071, 2008, which is incorporated herein by reference.

In some instances, transdermal delivery of one or more steroid hormones, or metabolites, modulators, or analogs thereof facilitated by an energy source may be combined with a method that perforates or abrades the skin of a subject. For example, a transdermal delivery method may combine iontophoresis with one or more microprojections that perforate the skin and enhance penetration and delivery of an agent as described, for example, in U.S. Pat. No. 6,835,184 and U.S. Patent Application 2006/0036209; which are incorporated herein by reference. In another example, an energy source such as iontophoresis or electroporation may be combined with electrically-induced ablation of skin cells as described in U.S. Pat. No. 7,113,821, which is incorporated herein by reference.

One or more steroid hormones, or metabolites, modulators, or analogs thereof may be delivered to a subject by a transdermal delivery method by one or more functional modes such as, for example, completely automatic with a preset dosage regimen, controlled by the subject or other individual, or automatically controlled by a feedback mechanism based the normal physiological level of the hormones. For example, a preset dosage regimen of exogenous hormones may be administered to a subject to supplement endogenous hormone levels to bring the latter to physiologically normal levels. As such, a transdermal delivery system may be designed which automatically times the activation and deactivation of an electrical power supply, for example, for delivery and cessation of delivery of a drug at a variable controlled rate at preset or preprogrammed time intervals as described in U.S. Pat. No. 5,224,928, which is incorporated herein by reference. The pre-set dosage regimen may be programmed into the transdermal delivery method at the time of manufacture. Alternatively, the transdermal delivery method may have a removable computer interface component that can be externally programmed for a specific drug delivery regimen and reinserted into the device such as described in U.S. Pat. No. 6,539,250, which is incorporated herein by reference.

In a further example, one or more steroid hormones, or metabolites, modulators, or analogs thereof may be delivered to a subject by a transdermal delivery method, parenteral delivery method, or oral or nasal delivery method by one or more functional modes, for example, automatically controlled by a feedback mechanism based on the normal physiological level of the hormones. Close control of steroid hormone levels significantly reduces complications in treatment or prevention of hormone-related diseases. A control method for the automation of steroid hormone infusion that utilizes emerging technologies in blood or tissue steroid hormone biosensors is presented. The controllers that have been developed provide tighter, more optimal control of blood or tissue steroid hormone levels, while accounting for variation in patient response, steroid hormone employed, or metabolite, modulator, or derivative thereof, and sensor bandwidth. Particular emphasis may be placed on controller simplicity and robustness necessary for medical devices and implants. In an example controlling blood glucose levels to treat a subject with type I diabetes, a PD controller with heavy emphasis on the derivative term is found to outperform the typically used proportional-weighted controllers in glucose tolerance and multi-meal tests. Dudde, et al., IEEE Trans Inf Technol Biomed. 10: 395-402, 2006, which is incorporated herein by reference. Suitability may be investigated of existing wearable continuous steroid hormone infusors controlled and adjusted by a control algorithm using continuous steroid hormone measurements as input to perform the functionality to maintain the normal physiological level of the hormones. Special attention may be given to the development of a continuous steroid hormone monitor and to evaluate which quality of input data is necessary for the control algorithm. Lam et al., Med Eng Phys. 24: 663-672, 2002, which is incorporated herein by reference.

In some instances, the delivery of one or more steroid hormones, or metabolites, modulators, or analogs thereof by a transdermal delivery method may be controlled either by the subject or other individual, for example, a healthcare provider, using on/off and/or high/low settings, for example, as described in U.S. Pat. No. 5,224,927, which is incorporated herein by reference. In some instances, it may be of benefit to limit or regulate the number of doses allowed by the subject. As such, the transdermal delivery method may incorporate a preprogrammed number of doses allowed during a given time period.

### Implantable Delivery Method

In general, a treatment regimen to maintain physiological cyclic levels of one or more steroid hormones, or modulators or metabolites thereof may be delivered systemically and/or to a specific site of action using an implantable delivery method. An implantable delivery method may incorporate a polymer or other matrix that allows for passive and slow release of one or more steroid hormones, or metabolites, modulators, or analogs thereof as exemplified, for example, by subcutaneous contraceptive implants. For example, a biologically active compound may be formulated with a solid hydrophilic polymer that swells by osmotic pressure after implantation, allowing interaction with a solubilizing agent and release of the biologically active compound through a non-porous rate-controlling membrane as described in U.S. Pat. No. 5,035,891, which is incorporated herein by reference. Alternatively, one or more steroid hormones, or metabolites, modulators, or analogs thereof may be delivered using an implantable delivery method that includes an infusion pump that actively moves the one or more steroid hormones from an associated reservoir into a subject. A variety of pumps may be incorporated into an implantable delivery method such as, for example, a piston pump, rotary vane pump, osmotic pump, Micro Electro Mechanical Systems (MEMS) pump, diaphragm pump, peristaltic pump, or solenoid piston pump. For example, the infusion pump may be a vapor-pressure powered pump in which a fluorocarbon charging fluid such as freon is used to drive the pump as a vapor-liquid mixture at normal body temperature and atmospheric pressure. Alternatively, the infusion pump may be a battery operated peristaltic pump. The latter is exemplified by an intrathecal drug delivery device in which an infusion pump with a controllable receiver unit is implanted under skin and a catheter is fed into the target site, in this case the spine (see, e.g., Belverud, Neurotherapeutics. 5:114-122, 2008, which is incorporated herein by reference). An external device may be used to wirelessly control the pump. The reservoir associated with the pump may be refillable via percutaneous injection.

A treatment regimen which includes one or more steroid hormones or metabolites, modulators, or analogs thereof for use in maintaining a substantially physiological level in a subject may be delivered using an implantable delivery method that incorporates a MEMS (Micro Electro Mechanical Systems) fabricated microchip. Examples of MEMS and/or microfabricated devices for potential delivery of a therapeutic agent are described in U.S. Pat. Nos. 5,993,414; 6,454,759; and 6,808,522, which are incorporated herein by reference. The MEMS implantable delivery method may have one or more microfabricated drug reservoirs such as, for example, microparticle reservoirs, silicon microarray reservoirs, and/or polymer microreservoirs as described by Grayson, et al., Proceedings of the IEEE, 92: 6-21, 2004, which is incorporated herein by reference. Microparticles fabricated from silicon may be used that contain an internal space which is loaded with drug using a microinjector and capped, for example, with a slow, dissolving gelatin or starch. Polymer microreservoirs may be fabricated by micromolding poly(dimethylsiloxane) or by patterning in multilayer poly(D-lactic acid) and (vinyl alcohol), for example. In some instances, the polymer microreservoirs may be capped with polymers that degrade at various rates in vivo depending upon the length of the polymer, allowing for controlled release of multiple doses.

Alternatively, an array of microreservoirs on a microchip may be used in which each dose of one or more steroid hormones, or metabolites, modulators, or analogs thereof is contained in its own reservoir and capped by an environmentally sensitive material. For example, the microreservoirs may be capped with a gold membrane which is weakened and ruptured by electrochemical dissolution in response to application of an anode voltage to the membrane in the presence of chloride ions, resulting in release of drug as described in U.S. Pat. No. 5,797,898 and in Prescott, et al., Nat. Biotech., 24:437-438, 2006, which are incorporated herein by reference. Alternatively, the microreservoirs may be capped by a temperature sensitive material that may be ruptured in response to selective application of heat to one or more of the reservoirs as described in U.S. Pat. No. 6,669,683, which is incorporated herein by reference. Wireless induction of a voltage or thermal trigger, for example, to a given reservoir of the microarray by a subject or other individual would enable on-demand release of one or more steroid hormones. Alternatively, the microchip array may incorporate a sensor component that signals release of one or more steroid hormones by a closed-loop mechanism in response to a chemical or physiological state as described in U.S. Pat. No. 6,976,982, which is incorporated herein by reference.

In some instances, the implantable delivery method may incorporate a natural and/or synthetic stimulus-responsive hydrogel or polymer which changes confirmation rapidly and reversibly in response to environmental stimuli such as, for example, temperature, pH, ionic strength, electrical potential, light, magnetic field or ultrasound (see, e.g., Stubbe, et al., Pharmaceutical Res., 21:1732-1740, 2004, which is incorporated herein by reference). Examples of polymers are described in U.S. Pat. Nos. 5,830,207; 6,720,402; and 7,033,571, which are incorporated herein by reference. In some instances, the one or more steroid hormones, or metabolites, modulators, or analogs thereof to be delivered by the implantable delivery method may be dissolved or dispersed in the hydrogel or polymer. Alternatively, a hydrogel and/or other stimulus-responsive polymer may be incorporated into an implantable delivery device. For example, a hydrogel or other polymer or other smart material may be used as an environmentally sensitive actuator to control flow of a therapeutic agent out of an implantable device as described in U.S. Pat. Nos. 6,416,495; 6,571,125; and 6,755,621, which are incorporated herein by reference. As such, an implantable delivery device may incorporate a hydrogel or other polymer that modulates delivery of one or more steroid hormones, or metabolites, modulators, or analogs thereof in response to environmental conditions.

In some instances, the implantable delivery method may be nonprogammable, delivering a predetermined dosage of one or more steroid hormones, or metabolites, modulators, or analogs thereof. For example, one or more steroid hormones may be administered using continuous infusion. Alternatively, the dosage of a one or more steroid hormones may be predetermined to deliver a dose based on a timing mechanism associated with the implantable device. For example, the timing device may be linked to the menstrual cycle and the established baseline levels of estrogen, progesterone, and testosterone, for example. Alternatively, the implantable device may be programmable, having on/off and/or variable delivery rates based on either external or internal control. External control may be mediated by manual manipulation of a hand-operated pulsative pump with one-way valves associated with a delivery device implanted near the surface of the skin, for example. Alternatively, external control may be mediated by remote control through an electromagnetic wireless signal such as, for example, infrared or radio waves that are able to trigger an electrical stimulus within the implanted device. Examples of remote control drug delivery devices are described in U.S. Pat. Nos. 5,928,195; 6,454,759; and 6,551,235, which are incorporated herein by reference. As such, one or more steroid hormones, or metabolites, modulators, or analogs thereof may be delivered by continuous infusion in response to an “on” trigger and stopped in response to an “off” trigger, for example. Alternatively, one or more steroid hormones, or metabolites, modulators, or analogs thereof may be delivered as a microbolus, for example, in response to an “on” trigger as described in U.S. Pat. No. 6,554,822, which is incorporated herein by reference. External control may be initiated by a caregiver. Alternatively, a subject may initiate delivery of one or more steroid hormones. As such, the system may have a built in mechanism to limit the number of allowable doses by a subject and/or caregiver in a given time frame as described, for example, in U.S. Pat. No. 6,796,956, which is incorporated herein by reference.

An implantable device for delivery of one or more steroid hormones or metabolites, modulators, or analogs thereof may be powered by a standard lithium battery. In some instances, the battery may be rechargeable. For example, a battery associated with an implantable device may be recharged transcutaneously via inductive coupling from an external power source temporarily positioned on or near the surface of the skin as described in U.S. Pat. No. 7,286,880, which is incorporated herein by reference. Alternatively, the energy source for an implantable device may come from within the subject. For example, an implantable device may be powered by conversion of thermal energy from the subject into an electrical current as described in U.S. Pat. No. 7,340,304, which is incorporated herein by reference. Other methods of recharging or directly driving a battery associated with an implantable device include but are not limited to electromagnetic energy transmission, piezoelectric power generation, thermoelectric devices, ultrasonic power motors, radio frequency recharging and optical recharging methods as described in Wei & Liu. Front. Energy Power Eng. China 2:1-13, 2008, which is incorporated herein by reference.

In some instances two or more steroid hormones, or metabolites, modulators, or analogs thereof may be administered using the same format. Alternatively, a combination of two or more modes of administration may be used for each dosing regimen. For example, a first hormone or metabolite, modulator, or analog thereof may be provided by transdermal administration and the a second hormone or metabolite, modulator, or analog thereof may be provided by vaginal administration. As another example, the a first hormone or metabolite, modulator, or analog thereof may be provided by oral administration, the a second hormone or metabolite, modulator, or analog thereof may be provided by transdermal administration, and a third hormone or metabolite, modulator, or analog thereof may be provided by transdermal administration.

Examples of compounds that may be used to as part of a treatment regimen administered by a transdermal delivery method, parenteral delivery method, and/or oral or nasal delivery method to maintain a physiological cyclic level of one or more steroid hormones include, but are not limited to, natural and synthetic compounds, metabolites, modulators, or analogs thereof. Compounds that may be used to alter estrogen levels, for example, include but are not limited to natural compounds with estrogenic activity such as estradiol (estradiol-17β), estriol, estrone, and their metabolites such as 2-hydroxyestrone, 2-methoxyestrone, 16α-hydroxyestrone, 17α-estradiol, 2-hydroxy-estradiol-17β, 2-methoxyl-estradiol-17β 6β-hydroxyl-estradiol-17β, 3-sulfate, 3-glucuronide, and 16-glucuronide; synthetic steroidal compounds having estrogenic activity such as estradiol 17β-acetate, estradiol 17β-cypionate, estradiol 17β-propionate, estradiol 3-benzoate, ethinyl estradiol, piperazine estrone sulfate, mestranol, and quinestrol; synthetic non-steroidal compounds having estrogenic activity such as diethylstilbestrol, chlorotrianisene, and methallenestril; and plant derived phytoestrogens having estrogenic activity such as coumestrol, 4′ methoxycoumestrol, repensol, trifoliol, daidzein, formononetin, genistein, and biochanin A. Esters, conjugates and prodrugs of suitable estrogens may also be used. Examples of estrogen prodrugs that may be used include, but are not limited to, estradiol acetate (which is converted in vivo to 17β-estradiol) and mestranol (which is converted in vivo to ethinyl estradiol). In some instances, a combination of estrogens may be used, see e.g. U.S. Pat. No. 6,911,438, which is incorporated herein by reference and provides a combination of three estrogens 2-hydroxyestrone, 17-β estradiol, and estriol, for example in a ratio determined by the method.

In some instances, the treatment regimen used to alter estrogen levels or their effects may include a selective estrogen receptor modulator (SERM). Examples of SERMs include by are not limited to tamoxifen, idoxifene, toremifene and raloxifene.

In some instances, the treatment regimen used to alter a hormone level, may be a natural precursor. For example, steroid hormone levels may be altered by providing a natural precursor such as, for example, testosterone, which may be converted in vivo to estradiol, or androstenedione, which may be convered to estrone or may be convered to testosterone. The treatment regimen might include a compound with enzymatic activity able to convert a naturally occurring precursor so as to alter a hormone level, for example a cytochrome P450 enzyme, or analog or modulator thereof. The treatment regimen might include modulating the activity of a resident enzyme, such as one active in steroidogenesis, by adding an inhibitor or activator.

Compounds that may be used as part of a treatment regimen to alter progesterone levels, for example, include but are not limited to natural and synthetic compounds having progestational activity, such as, for example, progesterone, levonorgestrel, norethindrone, norethindrone acetate, desogestrel, gestodene, dienogest, norgestimate, cyproterone acetate, norelgestromin, etonogestrel, ethynodiol diacetate, norgestrel, trimegestone, medroxyprogesterone acetate, chlormadinone acetate, drospirenone, and other natural and/or synthetic gestagens. Esters, conjugates, and prodrugs of suitable progestins may also be used. Additional compounds include metabolites and/or analogs of progesterone such as, for example, 20α-DH-P (4-pregnen-20α-ol-3-one), 5α-DH-P (5α-pregnan-3,20-dione), 3β,5α-TH-P (5α-pregnan-3b-ol-20-one), 20α-DH,5α-DH-P (5α-pregnan-20α-ol-3-one), 16α-OH—P (4-pregnen-16α-ol-3,20-dione), 5β-DH-P (5β-pregnan-3,20-dione), 20α-DH,3β,5α-TH-P (5α-pregnan-3β,20α-diol), 20α-DH, 3α,5α-TH-P (5α-pregnan-3α,20α-diol), 3α,5α-TH-P (5α-pregnan-3α-ol-20-one), 11α-OH—P (4-pregnen-11α-ol-3,20-dione), 11β-OH—P (4-pregnen-11β-ol-3,20-dione), 20α-DH,3α,5β-TH-P (5β-pregnan-3α,20α-diol), 17α-OH—P (4-pregnen-17α-ol-3-one), 17α-OH,20α-DH-P (4-pregnen-17,20α-diol-3-one) and 3α,5β-TH-P (5β-pregnan-3α-ol-20-one) (see, e.g., Quinkler, et al., Eur. J. Endocrinol. 146:789-800, 2002, which is incorporated herein by reference).

Compounds that may be used as part of a treatment regimen to alter testosterone and androgen levels, for example, include but are not limited natural androgens and metabolites thereof such as testosterone, dihydrotestosterone (DHT), dehydroepiandrosterone (DHEA), dehydroepiandrosterone sulfate (DHEAS), androstenedione, androst-5-ene-3β,17β-diol; synthetic androgens such as testosterone undecanoate, testosterone propionate, testosterone cypionate, testosterone enanthate, methyltestosterone, fluoxymesterone, oxymetholone, oxandrolone, nandrolone decanoate.

A treatment regimen to alter levels of one or more hormones may include compounds that stimulate the synthesis of one or more hormones. Such compounds may include gonadotropin hormones such as, for example, luteinizing hormone (LH) and follicle stimulating hormone (FSH), which modulate testosterone, estrogen and progesterone levels during the menstrual cycle. Examples of purified follicle stimulating hormone include but are not limited to urofollitropin (uFSH) purified from urine ofpostmenopausal women, recombinant forms of follicle stimulating hormone (rFSH) follitropin alfa and follitropin β. Examples of luteinizing hormone include recombinant human luteinizing hormone (rLH) lutropin.

### Pharmaceutical Formulations

The methods described herein maintain a substantially physiological cyclic level of one or more steroid hormones in a mammalian subject in need thereof which includes an individualized treatment regimen for the subject. The individualized treatment regimen includes replacement therapy for one or more steroid hormones, metabolites, modulators, or analogs thereof. The treatment regimen can be based upon information derived from pre-menopausal hormone levels or pre-disease hormone levels in the subject and current physiologic hormone levels. A treatment regimen includes a pharmaceutical formulation of one or more steroid hormones, or metabolites, modulators, or analogs thereof for use in maintaining a substantially physiological level in a subject. The pharmaceutical formulation may be formulated neat or may be combined with one or more acceptable carriers, diluents, excipients, and/or vehicles such as, for example, buffers, surfactants, preservatives, solubilizing agents, isotonicity agents, and stablilizing agents as appropriate. A “pharmaceutically acceptable” carrier, for example, may be approved by a regulatory agency of the state and/or Federal government such as, for example, the United States Food and Drug Administration (US FDA) or listed in the U.S. Pharmacopeia or other generally recognized pharmacopeia for use in animals, and more particularly in humans. Conventional formulation techniques generally known to practitioners are described in Remington: The Science and Practice of Pharmacy, 20^{th} Edition, Lippincott Williams & White, Baltimore, Md. (2000), which is incorporated herein by reference.

Acceptable pharmaceutical carriers include, but are not limited to, the following: sugars, such as lactose, glucose and sucrose; starches, such as corn starch and potato starch; cellulose, and its derivatives, such as sodium carboxymethyl cellulose, ethyl cellulose, cellulose acetate, and hydroxymethylcellulose; polyvinylpyrrolidone; cyclodextrin and amylose; powdered tragacanth; malt; gelatin, agar and pectin; talc; oils, such as mineral oil, polyhydroxyethoxylated castor oil, peanut oil, cottonseed oil, safflower oil, sesame oil, olive oil, corn oil and soybean oil; polysaccharides, such as alginic acid and acacia; fatty acids and fatty acid derivatives, such as stearic acid, magnesium and sodium stearate, fatty acid amines, pentaerythritol fatty acid esters; and fatty acid monoglycerides and diglycerides; glycols, such as propylene glycol; polyols, such as glycerin, sorbitol, mannitol and polyethylene glycol; esters, such as ethyl oleate and ethyl laurate; buffering agents, such as magnesium hydroxide, aluminum hydroxide and sodium benzoate/benzoic acid; water; isotonic saline; Ringer's solution; ethyl alcohol; phosphate buffer solutions; other non-toxic compatible substances employed in pharmaceutical compositions.

A treatment regimen including a pharmaceutical formulation of one or more steroid hormones or metabolites, modulators, or analogs thereof for use in maintaining a substantially physiological level may be formulated in a pharmaceutically acceptable liquid carrier. The liquid carrier or vehicle may be a solvent or liquid dispersion medium comprising, for example, water, saline solution, ethanol, a polyol, vegetable oils, nontoxic glyceryl esters, and suitable mixtures thereof. The solubility of a chemical blocking agent may be enhanced using solubility enhancers such as, for example, water; diols, such as propylene glycol and glycerol; mono-alcohols, such as ethanol, propanol, and higher alcohols; DMSO (dimethylsulfoxide); dimethylformamide, N,N-dimethylacetamide; 2-pyrrolidone, N-(2-hydroxyethyl) pyrrolidone, N-methylpyrrolidone, 1-dodecylazacycloheptan-2-one and other n-substituted-alkyl-azacycloalkyl-2-ones and other n-substituted-alkyl-azacycloalkyl-2-ones (azones). The proper fluidity may be maintained, for example, by the formation of liposomes, by the maintenance of the necessary particle size in the case of dispersions or by the use of surfactants. One or more antimicrobial agent may be included in the formulation such as, for example, parabens, chlorobutanol, phenol, sorbic acid, and/or thimerosal to prevent microbial contamination. In some instances, it may be preferable to include isotonic agents such as, for example, sugars, buffers, sodium chloride or combinations thereof.

A treatment regimen including a pharmaceutical formulation of one or more steroid hormones, or metabolites, modulators, or analogs thereof for use in maintaining a substantially physiological level may be formulated for transdermal delivery. For example, water-insoluble, stratum corneum-lipid modifiers such as for example 1,3-dioxanes, 1,3-dioxolanes and derivatives thereof, 5-, 6-, 7-, or 8-numbered lactams (e.g., butyrolactam, caprolactam), morpholine, cycloalkylene carbonate have been described for use in transdermal iontophoresis (see, e.g., U.S. Pat. No. 5,527,797, which is incorporated herein by reference). Other suitable penetration-enhancing agents include but are not limited to ethanol, hexanol, cyclohexanol, polyethylene glycol monolaurate, azacycloalkan-2-ones, linoleic acid, capric acid, lauric acid, neodecanoic acid hexane, cyclohexane, isopropylbenzene; aldehydes and ketones such as cyclohexanone, acetamide; N,N-di(lower alkyl)acetamides such as N,N-diethylacetamide, N,N-dimethyl acetamide; N-(2-hydroxyethyl)acetamide; esters such as N,N-di-lower alkyl sulfoxides; essential oils such as propylene glycol, glycerine, isopropyl myristate, and ethyl oleate; salicylates; and mixtures of any of the above (see, e.g., U.S. Patent Publication 2008/0119449).

In some instances, the treatment regimen including a pharmaceutical formulation of one or more steroid hormones or metabolites, modulators, or analogs thereof for use in maintaining a substantially physiological level may be formulated in a dispersed or dissolved form in a hydrogel or polymer associated with, for example, implantable or a transdermal delivery method. Examples of hydrogels and/or polymers include but are not limited to gelled and/or cross-linked water swellable polyolefins, polycarbonates, polyesters, polyamides, polyethers, polyepoxides and polyurethanes such as, for example, poly(acrylamide), poly(2-hydroxyethyl acrylate), poly(2-hydroxypropyl acrylate), poly(N-vinyl-2-pyrrolidone), poly(n-methylol acrylamide), poly(diacetone acrylamide), poly(2-hydroxylethyl methacrylate), poly(allyl alcohol). Other suitable polymers include but are not limited to cellulose ethers, methyl cellulose ethers, cellulose and hydroxylated cellulose, methyl cellulose and hydroxylated methyl cellulose, gums such as guar, locust, karaya, xanthan gelatin, and derivatives thereof. For iontophoresis, for example, the polymer or polymers may include an ionizable group such as, for example, (alkyl, aryl or aralkyl) carboxylic, phosphoric, glycolic or sulfonic acids, (alkyl, aryl or aralkyl) quaternary ammonium salts and protonated amines and/or other positively charged species as described in U.S. Pat. No. 5,558,633, which is incorporated herein by reference in its entirety.

Information regarding formulation of FDA approved steroid hormones, or metabolites, modulators, or analogs thereof may be found in the package insert and labeling documentation associated with each approved agent. A compendium of package inserts and FDA approved labeling may be found in the Physician's Desk Reference. Alternatively, formulation information for approved chemical blocking agents may be found on the interne at websites such as, for example, www.drugs.com and www.rxlist.com. For example, PREMARIN, an oral form of conjugated equine estrogens, contains active drug, calcium phosphate tribasic, hydroxypropyl cellulose, microcrystalline cellulose, powdered cellulose, hypromellose, lactose monohydrate, magnesium stearate, polyethylene glycol, sucrose, and titanium dioxide. For those steroid hormones or metabolites, modulators, or analogs thereof which do not currently have a formulation appropriate for use in any of the delivery methods described above, an appropriate formulation may be determined empirically and/or experimentally using standard practices. The pharmaceutical compositions are generally formulated as sterile, substantially isotonic and in full compliance with all Good Manufacturing Practice (GMP) regulations of the U.S. Food and Drug Administration.

### Kits

The invention provides kits comprising the compositions, e.g., nucleic acids, expression cassettes, vectors, cells, polypeptides (e.g., Scd1 polypeptides or toll-like receptor 2-signal activating polypeptides) and/or antibodies of the invention. The kits also can contain instructional material teaching the methodologies and uses of the invention, as described herein.

The methods and compositions are further described with reference to the following examples; however, it is to be understood that the methods and compositions are not limited to such examples.

### Exemplary Aspects

### Example 1

At least one treatment regimen for a subject including replacement therapy for one or more steroid hormones or metabolites or modulators thereof may be designed to maintain physiological premenopausal levels of one or more steroid hormones in a female subject with a family history of developing a chronic disease such as, for example, osteoporosis, Alzheimer's disease, colon cancer, or diabetes. In this context, a physiological level of a hormone includes the level of hormone measured at a given time. A physiological premenopausal level may be a level of the hormone as measured at a point in time during premenopause. A current physiological level may be the level of the hormone as measured just prior to determining a treatment regimen. The physiological levels of the one or more steroid hormones of the female subject may be provided by measurements collected just prior to determining a treatment regimen and/or provided as part of the subject's medical history. The physiological premenopausal levels may include cyclic and/or temporal, e.g., age-related or weight-related, variations. A treatment regimen may be determined based on the physiologic premenstrual levels and the current physiologic levels. The determined treatment regimen may include, for example, maintaining steroid hormone levels throughout perimenopause, menopause and/or postmenopause by administration of one or more exogenous steroid hormones, or metabolites, modulator, or analogs thereof and may include continual, cyclical, or time-dependent dosing as determined by the method as described herein.

The term menopause literally means the cessation of menstruation but it is commonly used to refer to the period in a woman's life when she passes out of her reproductive years. Menopause usually begins between the ages of 45 and 50, as the ovaries gradually cease to function and the production of the female sex hormones diminishes. The average age of menopause is 52 years of age. Perimenopause is a term used to describe the 5-15 years prior to the natural end of menstruation and is characterized by declining and fluctuating ovarian hormone production and as such is often associated with the physical symptoms of menopause such as hot flashes, increasing vaginal dryness, sleep problems, mood swings, and breast tenderness. Perimenopause typically begins between the ages of 35 and 50. The time period prior to perimenopause is referred to as premenopause.

Prior to determining a treatment regimen, additional information regarding the physiological status of the female subject may be gathered and assessed. For example, information on the subject's own history or her family's history of diseases, including genetic information, may be collected. Information gathering may include screening the subject for the presence of disease and/or undertaking genetic profile screening. For example, the subject might be screened for specific diseases or conditions and information used in the determining of the treatment regimen. For example, for individuals with a known history or who are at risk of certain cancers, for example epithelial cancers of the uterus, a treatment regimen may be determined using a progestin in addition to one or more estrogens in order to maintain the physiological levels of the steroid hormones. In a further embodiment, for a female subject with multiple risk factors for heart disease such as diabetes, high blood pressure, a strong family history or genetic predisposition to disease, a treatment regimen for maintaining a physiological level of one or more steroid hormones, may be determined that includes selecting a single specific form of estrogen or metabolite, modulator, mimetic, or analog thereof, and/or a particular means of delivery, such as transdermal, and/or also include providing an antagonistic tissue-specific estrogen receptor modulator (SERM) to inhibit responses. Selectivity of steroid hormones and receptors and its manipulation are discussed by Qiao, et al. in Gender Medicine. 5 Suppl. A, S46-S64, 2008, which is incorporated herein by reference.

To maintain a physiological level of one or more steroid hormones, the levels of endogenous hormones may be assessed. As part of the embodiment, endogenous hormones may be measured periodically during the years prior to perimenopause to establish a baseline or normal premenopausal level of steroid hormones for that individual. As such, testing may be performed as part of an annual exam. Preferably, the female subject would not be using hormone-based contraceptives at the time of testing as these may complicate measurement of natural/normal levels of hormones. If the subject uses hormone-based contraceptives, the subject is asked to stop using this form of contraception and use an alternative, non-hormonal form of contraception until testing is complete. Testing may be initiated following one or more normal menses when endogenous hormones are presumed to have returned to normal levels.

Hormones, including estrogen and progesterone, are known to fluctuate during the course of a normal menstrual cycle and to differ among women (see, e.g. Gandara, et al., Ann. N.Y. Acad. Sci. 1098: 446-450, 2007, Ellison et al., Lancet 342: 433-434, 1993; Pinheiro et al., Cancer Epidemiology Biomarkers & Prevention 14: 2147-2153, 2005; Nùñez-de la Mora et al., PLoS Med 4(5): e167 2007; Jasienka, et al., Cancer Epidemiology, Biomarkers and Prevention 15: 2131-2135, 2006; Small, et al., Human. Reproduction 20(8): 2162-2167, 2005; and Sharp et al., Am J Epidemiol 160: 729-740, 2004; which are incorporated herein by reference). For example, estrogen levels normally peak during the menstrual cycle at about day 15 during the follicular phase just prior to ovulation whereas progesterone levels peak at about day 25 in the luteal phase. Therefore, samples for hormone testing may be taken, for example, on multiple days over the course of one or more menstrual cycles. Because hormone levels may also fluctuate during the course of a 24 hour period, a specific time of day may be chosen for sample collection, for example, in early morning. In some instances, it may be of benefit to determine the hormonal fluctuations during the course of a 24 hour period. In this instance, sampling may be done multiple times during the course of a day and multiple days during the course of the menstrual cycle. In addition, hormone levels may undergo seasonal fluctuations, e.g., with higher levels recorded during the fall. As such, testing which is done on a yearly or biennial basis may be performed at the same time during the year. In addition, since physiologic changes may influence hormone production, testing may be performed over several years and/or when a health index changes, e.g., a change in body-mass index. At that time, additional samples may be tested and/or other data may be gathered such as the subject's weight. Steroid hormones, or metabolites or modulators thereof, that may be assayed in a bodily fluid or tissue to establish a physiologic level of hormone include, but are not limited to, estrogen fractions, for example, estrone [E1], estradiol (estradiol-17β, [E2]), and estriol [E3]; progesterone; androgens, for example, testosterone, dihydrotestosterone (DHT), dehydroepiandrosterone (DHEA), androstenedione, androst-5-ene-3β,17β-diol, and non-sterol hormones, for example, follicle stimulating hormone, luteinizing hormone, inhibin B, anti-Mullerian hormone, thyroid-related hormones and an estrogen receptor.

One or more blood samples may be collected everyday at the same time each day by a trained phlebotomist at a clinic or by a subject at home using a home blood sampling kit, e.g., as described in U.S. Pat. No. 5,938,679. Blood samples may be stored either in the refrigerator for up to 24 hours or in the freezer for longer periods of time, prior to testing. Alternatively or in addition, samples of saliva or cheek swabs may be collected for testing. Testing for the levels of hormones, for example estrogen and progesterone, may be carried out using a commercially available ELISA kit with hormone-specific immunoreagents and following the manufacturers instructions (from, e.g., Calbiotech, Spring Valley, Calif.; Estradiol ELISA catalog number ES071S, Progesterone ELISA catalog number PG072S). Estradiol is the form of estrogen most commonly measured in nonpregnant women, and may be measured and used in establishing an estrogen baseline during premenopause. However, estriol and estrone may also be measured during the establishment of the baseline and during further monitoring over the course of perimenopause and menopause. Estriol is known to increase during pregnancy and estrone is commonly measured following menopause. Two or more hormones may be measured so that their ratio can be calculated.

Alternatively, a radioimmunoassay, fluorescence immunoassay, or chemiluminescence immunoassay may be used to assess the steroid hormones, or metabolites or modulators thereof, and may depend upon the standard operating procedures of the clinical testing laboratory.

In some cases, the physiological premenopausal levels may be provided by an outside source. For example, a collection of physiological premenopausal levels may be provided as part of a patient's medical history.

In addition to assessing the physiological premenopausal levels, the current physiologic levels of one or more steroid hormones are assessed. These levels may be measured using any of the methods mentioned above, and may be a single measurement, or may include a collection of measurements taken, for example, over the course of one or more menstrual cycles or at more than one time of the day. In some cases, data regarding the current physiological levels may be provided by an outside source. For example one health care provider, such as a general practioner, might provide data to another user, e.g., a health care provider such as an endocrinologist.

The test results may be calculated or entered into a computer using a computer-readable medium. The computer would, for example, map out the monthly levels of steroid hormone and track changes in the monthly levels from one year to the next. As the levels of estrogens and progesterone decline from one testing period to the next, the computer program may calculate the difference between the current levels and baseline hormone levels and provide individualized dosage information for hormone replacement therapy to bring the current level of hormone up to the baseline physiologic level of hormone, maintaining the premenopausal level. For example, if the premenopausal level of estradiol of a female subject is approximately 75 pg/ml during the follicular phase of the menstrual cycle and falls to 50 pg/ml or below as the women enters perimenopause, sufficient estradiol or a metabolite may be administered to bring the blood level back to 75 pg/ml. She may also be prescribed progestin in doses specific to her needs. The dosing of each hormone is such that the total serum concentration, for example, of endogenous and exogenous hormone is equal to the baseline physiological concentration established during premenopause testing. For example, for a subject whose the baseline serum level of estradiol was measured at 70 pg/ml during the first week of her menstrual cycle, rose to over 200 pg/ml during ovulation and dropped back down to 70 pg/ml and for whom follow-up testing several years later determined the level of estradiol in the serum had fallen by 10%, sufficient exogenous'estradiol may be administered to replace the 10% and bring the serum levels back to the full baseline level. The amount of estradiol administered would depend, for example, upon the pharmacokinetics of the exogenous estradiol and the administration such as, for example, by oral, gel, transdermal or implanted route.

### Example 2

At least one treatment regimen for a subject including replacement therapy for one or more steroid hormones or metabolites or modulators thereof may be designed for treatment of a female subject following bilateral oophorectomy (or ovariectomy). Under normal pre-menopausal conditions, the ovaries produce a significant proportion of the circulating estrogens as well as approximately half of the circulating testosterone. The removal of the ovaries results in an abrupt decline in circulating hormones such as estrogens and testosterone, for example, and is associated with various symptoms of menopause as well as symptoms of hypoactive sexual desire disorder or loss of libido. As such, a treatment regimen may be designed to maintain the pre-oophorectomy hormone levels.

Prior to surgical intervention to remove the ovaries, information regarding the pre-oophorectomy levels of one or more steroid hormones or metabolites or modulators thereof may be collected to establish a pre-oophorectomy baseline. The pre-oophorectomy levels of the one or more steroid hormones or metabolites or modulators thereof of the female subject may be provided by collected measurements or provided as part of the subject's medical history. The pre-oophorectomy levels may include cyclic and/or temporal, e.g. age-related or weight-related, variations. The post-oophorectomy hormone levels may be collected on one or more occasions following surgery. A treatment regimen may be determined based on the pre-oophorectomy levels and the post-oophorectomy levels. The determined treatment regimen might, for example, include maintaining pre-oophorectomy hormone levels for a number of years by administration of one or more exogenous steroid hormones, or metabolites, modulators, mimetics, or analogs thereof, and may include continual, cyclical, or time-dependent dosing.

In some instances, the subject may provide medical information regarding her pre-oophorectomy levels of one or more steroid hormones or metabolites or modulators thereof. Alternatively, the subject may undergo testing to measure the levels of one or more steroid hormones or metabolites or modulators thereof to establish a pre-oophorectomy baseline. For example, estrogen and progesterone levels may be measured over the course of one or more menstrual cycles using the methods described herein. In some instances, testosterone levels may also be measured. Testosterone-levels, like estrogen and progesterone levels, fluctuate during the course of a normal menstrual cycle (see, e.g. Gandara, et al., Ann. N.Y. Acad. Sci. 1098:446-450, 2007; Sinha-Hinkim, et al., J. Clin. Endocrinol. Metab. 83:1312-1318, 1998, which are incorporated herein by reference). For example, testosterone levels reach peak levels on average 2-3 days prior to ovulation. As such, samples used for measuring pre-oophorectomy testosterone levels may be taken multiple days over the course of one or more menstrual cycles. Because hormone levels may also fluctuate during the course of a 24 hour period, a specific time of day may be chosen for sample collection such as, for example, early morning.

As an example, one or more blood samples may be collected multiple days over the course of one or more menstrual cycles either at home or at a clinic as described herein. Alternatively, one or more saliva samples or tissue swab may be used for measuring one or more hormones. See e.g., Biex, Inc., Dublin, Calif. For example, one or more saliva samples may be collected by a subject at home at the same time of day on multiple days over the course of one or more menstrual cycles (see, e.g., Gandara, et al., Ann. N.Y. Acad. Sci. 1098:446-450, 2007, which is incorporated herein by reference). At each sample collection, the subject spits into a 10 ml tube until a 2.5 ml sample of saliva is collected. The specimen is dated and stored in the subject's home freezer. All of the specimens collected during the course of one or two menstrual cycles are taken to a clinic for hormone testing. On the day of analysis, the samples are thawed and heated at 57° C. for 2 hours and centrifuged at 9,000×g for 4 minutes at 10° C. as described by Gandara, et al., 2007. Commercially available ELISA kits may be used to assess the levels of steroid hormones, for example the estrogens, progesterone, and/or testosterone (from, e.g., Pantex, Santa Monica, Calif.). Alternatively, a radioimmunoassay (e.g., using a kit from Diagnostic Systems Laboratories, Webster, Tex.), fluorescence immunoassay, or chemiluminescence immunoassay may be used, depending upon the standard operating procedures of the clinical testing laboratory.

Following surgical intervention to remove the ovaries, the levels of one or more steroid hormones, or metabolites or modulators thereof, may be re-assessed using the methods described herein to establish the post-oophorectomy hormone levels. Post-oopherectomy hormone levels may be measured at more than one time point after bilateral oophorectomy to monitor any additional changes in hormone levels. Information regarding pre-oopherectomy and post-oopherectomy hormone levels may be entered into a computer using a computer readable medium. A computer program may be used to calculate the change in pre-oopherectomy and post-oopherectomy hormone levels and accordingly aide in design of an individualized treatment regimen to maintain one or more steroid hormones at pre-oopherectomy levels as described herein. The treatment regimen as designed may vary during the course of a monthly cycle to mirror the pre-surgery, cyclical pattern of the subject. Information regarding changes over time in the post-oopherectomy hormone levels may be incorporated into the data set and used to design necessary adjustments in the treatment regimen.

An individualized regimen of estrogen and optionally progestin, or metabolites, modulators, mimetics, or analogs thereof, as appropriate, may be provided to the subject in the form of pills, patchs, gels, implants, or a combination thereof as described herein. An individualized regimen of testosterone may be provided to the female subject post-oophorectomy using, for example, a transdermal patch (see, e.g., Shifren, et al., N. Engl. J. Med. 343:682-688, 2000; Buster, et al., Obstet. Gynecol. 105:944-952, 2005, which are incorporated herein by reference). The patch may be formulated to deliver a daily dose of testosterone which achieves a serum level that mirrors the pre-oophorectomy level. The patch may be worn on the abdomen, for example, and changed every few days. Each patch may contain a dosage, for example, that is appropriate to that portion of the normal hormonal cycle as determined by the pre-oophorectomy baseline analysis. Alternatively, testosterone may be administered using a gel, an implant, a buccal tablet, or injection. In the case of an implant, the testosterone may be administered via a controllable pump, for example, which may be programmed to variably deliver sufficient testosterone either continuously or as a periodic bolus to achieve serum levels comparable with pre-oophorectomy levels.

### Example 3

At least one treatment regimen for a subject including replacement therapy for one or more steroid hormones or metabolites or modulators thereof may have benefit in reducing the risk of developing certain types of cancer, e.g., colorectal cancer, endometrial cancer, large bowel or rectal cancer, uterine cancer, ovarian cancer, or non-small cell lung cancer. Genetic profiling may be used to identify subjects at risk for developing certain types of cancer and may be incorporated into designing a treatment regimen. Prior to determining a treatment regimen, information on the subject's own history of cancer and/or the subject's family history of cancer, including genetic information, may be collected. The medical evaluation may include a genetic profile of the subject regarding genes, genetic mutations, or genetic polymorphisms that may indicate risk factors associated with developing one or more cancer types. A physician may use the genetic profiling or genetic testing information to determine a genetic basis for needed treatment to maintain a substantially physiological cyclic level of one or more steroid hormones, or metabolites or modulators thereof, in a subject in need thereof.

As an example, a treatment regimen to maintain physiological cyclic levels of one or more steroid hormones or metabolites or modulators thereof, may be designed for a subject at increased risk of developing non-small cell lung carcinoma (NSCLC). Hormone therapy may lower the risk of developing NSCLC, particularly in former cigarette smokers who are at increased risk of developing lung cancer (see, e.g., Ramnath, et al., Oncology 73:305-310, 2007, which is incorporated herein by reference). As such, information regarding a subject's relative risk of developing NSCLC, for example, or any other cancer type, may be collected and used in combination with information regarding a subject's premenopausal and current levels of one or more hormones or metabolites thereof to design a treatment regimen.

Information may be gathered regarding a subject's family history of lung cancer as well as information regarding a subject's lifestyle choices, including but not limited to, occupational history (exposure to fossil fuel-derived substances), cigarette smoking, and dietary habits, for example. For example, information may also be gathered regarding a subject's genetic profile as it relates to NSCLC and other cancers.

Genetic profiling can identify subjects at risk for developing certain types of cancer. Genetic profiling can identify polymorphisms in specific genes have been linked with increased risk of developing specific cancers. Information regarding a subject's genetic profile and relative risk of developing a cancer such as NSCLC, for example, may be provided by the subject. Alternatively, this information may be collected by performing a genetic profile on the subject's DNA. Genomic DNA may be isolated from any of a number of bodily fluids or tissues such as, for example, blood, urine, saliva, tissue biopsies, and/or cheek scrapes. For example, genomic DNA may be isolated from whole blood by lysis of the blood components and isolation of the released genomic DNA by immobilization in a precipitate or on beads, membranes or other appropriate substrate using a commercially available kit (from, e.g., Invitrogen, Carlsbad Calif.; Qiagen, Valencia, Calif.; Stratagene, La Jolla, Calif.).

A subject's isolated genomic DNA may be used for amplification of specific genomic sequences to detect specific mutations or polymorphisms that may be linked to increased risk of developing a cancer. For example, polymerase chain reaction (PCR) assays may be combined with restriction fragment length polymorphism (RFLP) assays or DNA sequencing to detect specific mutations or polymorphisms. In the case of NSCLC, polymorphisms in three DNA repair genes, xeroderma pigmentosum complementation group D (XPD), X-ray repair cross-complementing group 1 (XRCC1), and X-ray repair cross-complementing group 3 (XRCC3), have been linked to increased risk of developing NSCLC (see, e.g., Butkiewicz, et al., Carcinogenesis. 22:593-597, 2001, which is incorporated herein by reference). As such, a subject's DNA may be analyzed for the presence of one or more of these polymorphisms. PCR primers directed against XPD, XRCC1, and XRCC3 may be designed as described in Shen, et al. and combined with the subject's genomic DNA for amplification (Shen, et al., Cancer Res. 58:604-608, 1998, which is incorporated herein by reference). For example, DNA amplification may be carried out using the following thermal cycling conditions: denaturation, 94° C. for 4 minutes followed by 35-40 cycles of denaturation at 94° C. for 30 seconds; primer annealing at 55-64° C. (depending upon the properties of the PCR primers) for 30 seconds; primer extension at 72° C. for 30 seconds; and a final extension at the end of amplification at 72° C. for 4 minutes. The resulting amplification products may be directly sequenced to determine the presence or absence of mutations or polymorphisms using standard procedures. Alternatively, the amplification products may be treated with specific restriction enzymes known to generate differential end products in the presence or absence of specific mutations or polymorphisms which may be visualized, for example, using agarose gel electrophoresis and ethidium bromide. The resulting information regarding the presence or absence of specific mutations and/or polymorphisms may be added to the subject's medical history.

A physician may use the genetic profiling information and the subject's medical history in combination with information regarding a subject's physiological premenopausal hormone levels and current physiological hormone levels to design a treatment regimen to maintain a substantially physiological cyclic level of one or more hormones. Information regarding a subject's physiological premenopausal hormone levels and/or current physiological hormone levels may be provided in the subject's medical history or may be collected as described herein. Information regarding a subject's genetic profile and medical history as well as current and premenopausal hormone levels may be entered into a computer using a computer readable medium. A computer program may be used to monitor changes in the monthly hormone levels from one measurement period to the next. Computational analysis may be used to design an individualized treatment regimen based on the pre-menopausal and current hormone levels and the genetic profiling information.

### Example 4

At least one treatment regimen for a subject including replacement therapy for one or more steroid hormones or metabolites or modulators thereof in a subject may be designed to prevent development of late onset dementia and neurodegeneration associated, for example, with Alzheimer's disease, Parkinson's disease, and amyotrophic lateral sclerosis (ALS). Several studies suggest that age-related declines in estrogen and testosterone levels are associated with decreased cognitive ability and increased risk of developing Alzheimer's disease, Parkinson's disease, and amyotrophic lateral sclerosis (ALS) (see, e.g., Morrison, et al., J. Neurosci. 26:10332-10348, 2006; Ryan, et al., Int. Psychogeriatr. 20:47-56, 2008; McLay, et al., J. Neuropsychiatry Lin. Neurosci. 15:161-167, 2003; and Beauchet, Eur. J. Endocrinol. 155:773-781, 2006, which are incorporated herein by reference). As such, maintaining physiological levels of one or more steroid hormones, or metabolites or modulators thereof may have benefit in attenuating or preventing one or more neurodegenerative disorders.

A treatment regimen for maintaining a physiological level of one or more steroid hormones, or metabolites or modulators thereof, may be designed based on a subject's medical history regarding her relative risk of developing a neurodegenerative disorder. In some instances, a subject may have a known family history regarding development of a neurodegenerative disorder and this information becomes part of the subject's medical history. Alternatively, a subject's medical history may include a genetic profile in which polymorphisms associated with neurodegenerative disorders have previously been identified. For example, a subject may have previously been tested for the presence of specific polymorphisms in the apolipoprotein E (APOE) gene, one allele of which has been linked to an increased risk of developing Alzheimer's disease (see, e.g., Strittmatter, et al., Proc. Natl. Acad. Sci., USA., 90:1977-1981, 1993, which is incorporated herein by reference). In some instances, information regarding a subject's risk for developing a neurodegenerative disorder may be unknown. As such, genetic profiling may be performed to determine relative risk. For example, a subject may undergo APOE genotyping. A common method for APOE genotyping involves amplification by PCR of a 244 base-pair fragment within exon 4 of the APOE gene followed by digestion of the PCR fragment with the endonuclease HhaI, creating a characteristic pattern of DNA bands for each of the three common APOE alleles upon gel electrophoresis (see, e.g., Hegele. Clin Chem. 45: 1579-1580, 1999, which is incorporated herein by reference). In particular, the APOEε4 allele is strongly associated with Alzheimer's disease with the sensitivity ranging from 46 to 78 percent and the specificity reaching nearly 100 percent (see, e.g., Mayeux, et al., N. Engl. J. Med. 338:506-511, 1998, which is incorporated herein by reference).

Information regarding a female subject's relative risk of developing a neurodegenerative disorder may be combined with information regarding her physiological premenopausal hormone levels and her current physiological hormone levels to design a treatment regimen for maintaining a physiological level of one or more steroid hormones or metabolites or modulators thereof. Information regarding a subject's physiological premenopausal hormone levels and/or current physiological hormone levels may be provided as part of the subject's medical history or may be provided by collected measurements as described herein. Information regarding current and premenopausal hormone levels as well as risk factors for developing a neurodegenerative disorder may be entered into a computer using a computer readable medium. A computer program may be used to monitor changes in the monthly hormone levels from one year to the next and to aide in design of an individualized treatment regimen as described herein.

In some instances, a treatment regimen for maintaining a physiological level of one or more steroid hormones, or metabolites or modulators thereof, to prevent neurodegenerative disorders may be designed for a male subject. For example, declining endogenous levels of testosterone in aging men have been linked to reduced cognition, and administration of exogenous testosterone may improve some aspects of cognitive ability (see, e.g., Beauchet, Eur. J. Endocrinol. 155:773-781, 2006, which is incorporated herein by reference). As such, a treatment regimen may be designed which provides a pharmaceutical composition including testosterone, or metabolites, modulators, or analogs thereof to a level that compensates for the loss of endogenous testosterone due to aging. Information regarding past and current physiological levels of testosterone in a male subject may be provided by collected measurements or provided as part of the subject's medical history. In some instances, it may be beneficial to collect information regarding the levels of testosterone in a male subject prior to the age of 40, a point at which normal testosterone levels may begin to decline (see, e.g., Feldman, et al., J. Clin. Endocrinol. Metab. 87:589-598, 2002, which is incorporated herein by reference). Information regarding the levels of testosterone in a male subject may be collected just prior to designing a treatment regimen to establish a subject's current physiological level of testosterone and may continue to be collected on a periodic basis to monitor changes in endogenous testosterone levels over time. To establish information regarding testosterone levels in a male subject, measurements may be collected on an hourly, daily, monthly and/or yearly basis, consistent with normal fluctuations in male testosterone levels (see, e.g. Hirschenhauser, et al., Hormones Behavior 42:172-181, 2002; Svartberg, et al., J. Clin. Endocrinol. Metab. 88:3099-3104, 2003, which are incorporated herein by reference). For example, one or more blood samples may be collected at the same time of day for multiple days at a clinic or at home over the course of one or two months. Alternatively, saliva samples may be collected at home every morning upon rising, for example, to standardize variation due to time of day and to avoid contamination of saliva samples with food or toothpaste, for example. Testosterone levels in samples collected from a male subject may be measured using an immunoassay, for example, as described herein.

Information collected from a male subject regarding current physiological levels of testosterone and physiological levels of testosterone prior to age 40, for example, may be entered into a computer using a computer readable medium that maps out the normal fluctuations in testosterone levels and tracks changes in the normal fluctuations from one year to the next. Information regarding testosterone levels of a male subject may be combined with a subject's medical history, family history and/or genotyping for neurodegenerative disorders to design a treatment regimen. As the level of testosterone from one collection period to the next declines, the computer program may calculate the difference between the current levels and pre-40 testosterone levels, for example, and may design a treatment regimen that brings the current level of testosterone up to the pre-age 40 level of testosterone, for example. Periodic adjustments in the treatment regimen may be made based on updated information regarding the current testosterone levels of a male subject relative to the pre-age 40 testosterone levels.

### Example 5

At least one treatment regimen for a female subject including replacement therapy for one or more steroid hormones or metabolites or modulators thereof in a subject may be designed based on genetic profiling information wherein the determination is based on one or more polymorphisms in the estrogen receptor gene sequences. Alternatively, a treatment regimen may be based on a combination of the genetic profiling information and on measured levels of steroid hormones or metabolites or modulators thereof in the subject. In one instance, a subject with a specific polymorphism in the estrogen receptors may differentially respond to a treatment regimen designed to maintain premenopausal steroid hormone levels relative to a subject lacking a specific polymorphism. As an example, the specific estrogen-receptor a polymorphism IVS1-401 C/C genotype is associated with increased levels of high density lipoprotein (HDL) cholesterol in response to estrogen and progestin treatment (see, e.g., U.S. Pat. No. 6,828,103; Herrington, et al., N. Engl. J. Med. 346:967-974, 2002, which are incorporated herein by reference). Increased HDL cholesterol levels are associated with lowered cardiovascular risk. As such, maintaining a physiological level of estrogen in a female subject with the IVS1-401 C/C genotype, for example, may provide a cardiovascular benefit.

Genetic profiling information regarding mutations and/or polymorphisms in the estrogen receptor a and/or estrogen receptor β genes of a subject's genomic DNA may be provided by the medical history of the subject. Alternatively, this information may be collected by performing a genetic profile on the subject's DNA prior to administering a treatment regimen. Genomic DNA may be isolated from any of a number of bodily fluids or tissues and purified using commercially available kits as described herein. A subject's isolated genomic DNA may be used for amplification of specific genomic sequences to detect specific mutations or polymorphisms in the estrogen receptor a and/or estrogen receptor β genes. For example, PCR amplification may be combined with RFLP assays or DNA sequencing as described herein to detect specific mutations or polymorphisms. Primers specific for amplification of DNA containing the IVS1-401 C/C genotype, for example, may be generated as described in U.S. Pat. No. 6,828,103, which is incorporated herein by reference, and used to generate PCR amplification fragments. DNA sequencing of the PCR amplification fragments may be used to identify the IVS1-401 C/C genotype relative to other genotypes at this locus. Information collected regarding the presence or absence of the IVS1-401 C/C genotype may be taken into consideration when designing a treatment regimen including replacement therapy for the one or more steroid hormones or metabolites or modulators thereof in a method for maintaining a physiological premenopausal level of one or more steroid hormones in the subject.

Additional physiological information may be collected in conjunction with genetic profiling. For example, in the context of the estrogen receptor a genotype IVS1-401 C/C, information regarding HDL cholesterol levels of a subject may be collected and used in combination with information regarding estrogen receptor genotyping and premenopausal and current hormone levels to design a treatment regimen. Information regarding HDL cholesterol levels may be provided as part of the subject's medical history. Alternatively, information regarding a subject's HDL cholesterol levels may be provided by collected measurements. HDL cholesterol, along with LDL cholesterol and triglycerides may be measured in a fasted, morning blood draw from a subject using autoanalyzer (for example, Technicon RA-1000® random-access chemistry analyzer, Diamond Diagnostics, Holliston, Mass.) as described in Herrington, et al., N. Engl. J. Med. 346:967-974, 2002, which is incorporated herein by reference. Information regarding HDL cholesterol levels of a subject may be collected prior to initiation of a treatment regimen to establish a pre-treatment baseline. Following initiation of a treatment regimen, HDL cholesterol levels of a subject may be collect on a periodic basis to assess the benefits of the treatment regimen.

The estrogen receptor genetic profiling information and physiological information of a subject may be used in conjunction with information regarding her physiological premenopausal hormone levels and her current physiological hormone levels to design a treatment regimen including replacement therapy for the one or more steroid hormones or metabolites or modulators thereof in a method for maintaining a physiological premenopausal level of one or more steroid hormones in the subject. Information regarding a subject's physiological premenopausal hormone levels and/or current physiological hormone levels may be provided as part of the subject's medical history or may be provided by collected measurements as described herein. The estrogen receptor genetic profiling information, physiological information, and current and premenopausal hormone levels may be entered into a computer using a computer readable medium. A computer program may be used to monitor changes in the monthly hormone levels from one year to the next and to aide in the initial design of an individualized treatment regimen and to aide in design of appropriate adjustments in an individualized treatment regimen as described herein.

Each recited range includes all combinations and sub-combinations of ranges, as well as specific numerals contained therein.

All publications and patent applications cited in this specification are herein incorporated by reference to the extent not inconsistent with the description herein and for all purposes as if each individual publication or patent application were specifically and individually indicated to be incorporated by reference for all purposes.

Those having ordinary skill in the art will recognize that the state of the art has progressed to the point where there is little distinction left between hardware and software implementations of aspects of systems; the use of hardware or software is generally (but not always, in that in certain contexts the choice between hardware and software can become significant) a design choice representing cost vs. efficiency tradeoffs. Those having ordinary skill in the art will appreciate that there are various vehicles by which processes and/or systems and/or other technologies described herein can be effected (e.g., hardware, software, and/or firmware), and that the preferred vehicle will vary with the context in which the processes and/or systems and/or other technologies are deployed. For example, if an implementer determines that speed and accuracy are paramount, the implementer may opt for a mainly hardware and/or firmware vehicle; alternatively, if flexibility is paramount, the implementer may opt for a mainly software implementation; or, yet again alternatively, the implementer may opt for some combination of hardware, software, and/or firmware. Hence, there are several possible vehicles by which the processes and/or devices and/or other technologies described herein may be effected, none of which is inherently superior to the other in that any vehicle to be utilized is a choice dependent upon the context in which the vehicle will be deployed and the specific concerns (e.g., speed, flexibility, or predictability) of the implementer, any of which may vary. Those skilled in the art will recognize that optical aspects of implementations will typically employ optically-oriented hardware, software, and or firmware.

In a general sense, those skilled in the art will recognize that the various aspects described herein which can be implemented, individually and/or collectively, by a wide range of hardware, software, firmware, or any combination thereof can be viewed as being composed of various types of “electrical circuitry.” Consequently, as used herein “electrical circuitry” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of random access memory), and/or electrical circuitry forming a communications device (e.g., a modem, communications switch, or optical-electrical equipment). Those having ordinary skill in the art will recognize that the subject matter described herein may be implemented in an analog or digital fashion or some combination thereof.

The herein described components (e.g., steps), devices, and objects and the description accompanying them are used as examples for the sake of conceptual clarity and that various configuration modifications using the disclosure provided herein are within the skill of those in the art. Consequently, as used herein, the specific exemplars set forth and the accompanying description are intended to be representative of their more general classes. In general, use of any specific exemplar herein is also intended to be representative of its class, and the non-inclusion of such specific components (e.g., steps), devices, and objects herein should not be taken as indicating that limitation is desired.

With respect to the use of substantially any plural or singular terms herein, those having skill in the art can translate from the plural to the singular or from the singular to the plural as is appropriate to the context or application. The various singular/plural permutations are not expressly set forth herein for sake of clarity.

The herein described subject matter sometimes illustrates different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected,” or “operably coupled,” to each other to achieve the desired functionality, and any two components capable of being so associated can also be viewed as being “operably couplable,” to each other to achieve the desired functionality. Specific examples of operably couplable include but are not limited to physically mateable or physically interacting components or wirelessly interactable or wirelessly interacting components or logically interacting or logically interactable components.

While particular aspects of the present subject matter described herein have been shown and described, it will be apparent to those skilled in the art that, based upon the teachings herein, changes and modifications may be made without departing from the subject matter described herein and its broader aspects and, therefore, the appended claims are to encompass within their scope all such changes and modifications as are within the true spirit and scope of the subject matter described herein. Furthermore, it is to be understood that the invention is defined by the appended claims. It will be understood that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an”; the same holds true for the use of definite articles used to introduce claim recitations. In addition, even if a specific number of an introduced claim recitation is explicitly recited, such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, or A, B, and C together, etc.). Virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

The various aspects and embodiments disclosed herein are for purposes of illustration and are not intended to be limiting, with the true scope and spirit being indicated by the following claims.

What is claimed is:

1.-59. (canceled)

60. A method for treating a metabolic disease in a mammalian subject comprising:
providing to the subject at least one treatment regimen including replacement therapy for the one or more steroid hormones or metabolites or modulators thereof, wherein the at least one treatment regimen is determined based on the subject's pre-menopausal cyclic steroid hormone levels and on current cyclic steroid hormone levels.

61. The method of claim 60, wherein the at least one treatment regimen is configured to maintain the subject's one or more steroid hormones or metabolites or modulators thereof at substantially physiological cyclic pre-menopausal levels in an amount effective to reduce or eliminate metabolic disease in the subject.

62. The method of claim 60, wherein the current cyclic steroid hormone levels are based on one or more of peri-menopausal, early menopausal, late menopausal, or post-menopausal steroid hormone levels of the subject.

63. The method of claim 61, wherein the metabolic disease includes at least one of diabetes, metabolic syndrome, or thyroid disease.

64.-109. (canceled)

110. The method of claim 60, further comprising determining the one or more steroid hormones levels in the subject during a treatment period.

111. The method of claim 110, wherein the treatment period includes a time period preceding treatment or a time period during treatment with the at least one treatment regimen.

112. The method of claim 110, wherein the determining the one or more steroid hormones levels occurs at multiple time points during the treatment period.

113. The method of claim 110, further comprising providing to the subject at least one second treatment regimen adjusted to maintain the subject's one or more steroid hormones or one or more metabolites or modulators thereof at substantially physiological cyclic pre-menopausal levels.

114. The method of claim 60, wherein the at least one treatment regimen is determined based at least in part on a time-history of serum steroid hormone levels in the subject, on inferred peak values or minimal values of serum steroid hormone levels in the subject, on age of the subject, or on categorization relative to profiles of patient populations.

115. The method of claim 60, wherein the at least one treatment regimen is determined based at least in part on Fourier analysis of the cyclic steroid hormone levels in the subject, or on harmonic analysis of the cyclic steroid hormone levels in the subject.

116. The method of claim 60, wherein the at least one treatment regimen is determined based on a genetic profile of the subject.

117. The method of claim 60, further comprising determining a genetic profile of the subject.

118. The method of claim 60, wherein the at least one treatment regimen is determined based at least in part on scaled values of the pre-menopausal cyclic steroid hormone levels in the subject.

119. The method of claim 118, wherein the at least one treatment regimen is determined based at least in part on a scaled value approximately equal to one.

120. The method of claim 118, wherein the at least one treatment regimen is determined based at least in part on a scaled value dependent on age of the subject.

121. The method of claim 60, wherein the subject is peri-menopausal, early menopausal, late menopausal, or post menopausal.

122. The method of claim 60, further comprising determining the subject's one or more steroid hormones levels when the subject is premenopausal and has no underlying condition affecting production of steroid hormones.

123. The method of claim 122, wherein the at least one treatment regimen is determined based at least in part on a time-history of serum steroid hormone levels in the subject, on inferred peak values or minimal values of serum steroid hormone levels in the subject, on age of the subject, or on categorization relative to profiles of patient populations.

124. The method of claim 122, wherein the at least one treatment regimen is determined based on a genetic profile of the subject.

125. The method of claim 122, further comprising determining a genetic profile of the subject.

126. The method of claim 60, wherein the at least one treatment regimen is determined while the subject is not pre-menopausal.

127. The method of claim 60, wherein the at least one treatment regimen is determined while the subject has an underlying condition affecting production of hormones.

128. The method of claim 127, wherein the subject has undergone oopherectomy, and the at least one treatment regimen is determined based on the subject's pre-surgical cyclic steroid hormone levels.

129. The method of claim 60, wherein the subject is female.

130. The method of claim 60, wherein the treatment regimen is provided orally.

131. The method of claim 60, wherein the treatment regimen is provided transdermally or subdermally.

132. The method of claim 60, wherein the hormones or their metabolically related compounds are sterols, androgens, testosterone, progestogens, estrogens, estradiol, estrone, follicle-stimulating hormone, luteinizing hormone, inhibin B, anti-Mullerian hormone thyroid-related hormones, or analogs or derivatives thereof.

133. The method of claim 132, wherein the androgen is testosterone.

134. The method of claim 132, wherein the estrogen is an estradiol or estrone.

135. The method of claim 60, wherein the at least one treatment regimen is determined based at least in part on one or more ratios of levels of the steroid hormones or metabolically related compounds in the subject, and the treatment regimen restores the one or more ratios to physiological cyclic pre-menopausal levels.

136. The method of claim 135, wherein the one or more ratios include a ratio of different estradiols, an estradiol to an estrone, or a testosterone to an estrogen.

137. The method of claim 135, wherein the one or more ratios include a ratio of the one or more steroid hormones or metabolically related compounds at different time intervals of the cycle in the subject.

138. The method of claim 60, further comprising determining one or more ratios of levels of the steroid hormones or metabolically related compounds in the subject.

139. The method of claim 60, wherein the at least one treatment regimen is determined based on the subject's pre-menopausal cyclic levels of one or more steroid hormone receptors and on the subject's current levels of the one or more steroid hormone receptors.

140. The method of claim 139, wherein the one or more hormone receptors is an estrogen receptor.

141. The method of claim 60, wherein the at least one treatment regimen is based on diagnostic data.

142. The method of claim 141, wherein the diagnostic data is based on serum levels of at least one of inflammation-related compounds, lipid-related compounds, or disease-related markers.

143. The method of claim 142, wherein the at least one inflammation-related compound is C-reactive protein.

144. The method of claim 142, wherein the at least one lipid-related compound includes a high density lipoprotein or a low density lipoprotein.

145. The method of claim 142, wherein the at least one disease-related marker includes a metabolic disease marker.

146. The method of claim 60, wherein the at least one treatment regimen is determined using an algorithm designed to determine a dosage of the one or more steroid hormones or metabolites, modulators, or analogs thereof in the at least one treatment regimen.

147. The method of claim 60, wherein the subject's pre-menopausal cyclic steroid hormone levels or one or more of the subject's peri-menopausal, early menopausal, late menopausal, or post-menopausal steroid hormone levels are determined for a period of time from at least approximately 7 days to approximately 168 days.
